(19) United States
(12) Patent Application Publication
(10) Pub. No.: US 2022/0168575 A1
(43) Pub. Date: Jun. 2, 2022

(54) DEVICE AND METHOD FOR ATRIAL TACHYARRHYTHMIA DETECTION

(71) Applicant: Medtronic, Inc., Minneapolis, MN (US)

(72) Inventors: Saul E. GREENHUT, Denver, CO (US); Vincent P. GANION, Blaine, MN (US); Yanina GRINBERG, Plymouth, MN (US); Alexander R. MATTSON, St. Paul, MN (US)

(21) Appl. No.: 17/515,895

(22) Filed: Nov. 1, 2021

Related U.S. Application Data
(60) Provisional application No. 63/119,016, filed on Nov. 30, 2020.

(51) Int. Cl. A61N 1/365; A61N 1/362; A61N 1/37; A61N 1/08

(52) U.S. Cl. CPC A61N 1/36578; A61N 1/3624; A61N 1/08; A61N 1/3655; A61N 1/3706

(57) ABSTRACT

A medical device is configured to sense an acceleration signal and determine at least one frequency metric from the acceleration signal that is correlated to a frequency of oscillations of the acceleration signal. The medial device is configured to determine that the at least one frequency metric meets atrial tachyarrhythmia criteria and detect an atrial tachyarrhythmia in response to at least the frequency metric meeting the atrial tachyarrhythmia criteria.

### REFERENCE TO RELATED APPLICATIONS

This application claims the benefit of the filing date of provisional U.S. Application Ser. No. 63/119,016, filed Nov. 30, 2020, the content of which is incorporated herein by reference in its entirety.

### TECHNICAL FIELD

This disclosure relates to a medical device and method for detecting atrial tachyarrhythmia

### BACKGROUND

During normal sinus rhythm (NSR), the heartbeat is regulated by electrical signals produced by the sino-atrial (SA) node located in the right atrial wall. Each atrial depolarization signal produced by the SA node spreads across the atria, causing the depolarization and contraction of the atria, and arrives at the atrioventricular (AV) node. The AV node responds by propagating a ventricular depolarization signal through the bundle of His of the ventricular septum and thereafter to the bundle branches and the Purkinje muscle fibers of the right and left ventricles, sometimes referred to as the “His-Purkinje system.”

Patients with a conduction system abnormality, e.g., SA node dysfunction or poor AV node conduction, bundle branch block, or other conduction abnormalities, may receive a pacemaker to restore a more normal heart rhythm. A single chamber pacemaker coupled to a transvenous lead carrying electrodes positioned in the right atrium may provide atrial pacing to treat a patient having SA node dysfunction. Intracardiac pacemakers have been introduced or proposed for implantation entirely within a patient's heart eliminating the need for transvenous leads. For example, an atrial intracardiac pacemaker may provide sensing and pacing from within an atrial chamber of a patient having bradycardia or SA node dysfunction. When the AV node is functioning normally, single chamber atrial pacing may sufficiently correct the heart rhythm. The pacing-evoked atrial depolarizations may be conducted normally to the ventricles via the AV node and the His-Purkinje system maintaining normal AV synchrony.

Atrial tachyarrhythmias are atrial rhythms that may arise from a non-sinus node location, and occur with a relatively high rate of incidence, even in a patient having an atrial pacemaker. Atrial fibrillation may be the most common form of arrhythmia. Non-sinus atrial tachycardia (AT) and atrial fibrillation (AF) can lead to serious and life-threatening complications, including blood clots, stroke, heart failure and more serious arrhythmias. Atrial tachyarrhythmias, while highly prevalent, tend to be underdiagnosed and undertreated.

### SUMMARY

The techniques of this disclosure generally relate to a medical device configured to sense an acceleration signal from an atrial location and detect atrial tachyarrhythmia based on an analysis of the acceleration signal. The medical device is an atrial pacemaker in some examples and may be implantable wholly in an atrial chamber. The medical device analyzes the acceleration signal to determine a frequency metric from the acceleration signal that is correlated to the frequency of oscillations of the acceleration signal. Atrial tachyarrhythmia may be detected based on the frequency metric. The medical device may sense an atrial electrical signal and detect a fast atrial rate based on sensing atrial event signals. In some examples, upon detecting a fast atrial rate from the atrial electrical signal, the medical device analyzes the acceleration signal. Atrial tachyarrhythmia detection criteria applied by a control circuit of the medical device may require that the frequency metric be indicative of a frequency of oscillations of the acceleration signal that is greater than a frequency of sensed atrial event signals. The medical device may detect atrial tachyarrhythmia in response to both the acceleration signal meeting atrial tachyarrhythmia criteria and an atrial electrical event rate, determined from a cardiac electrical signal, meeting atrial tachyarrhythmia rate criteria.

In one example, the disclosure provides a medical device including an accelerometer configured to sense an acceleration signal and a control circuit configured to receive the acceleration signal. The control circuit is configured to determine at least one frequency metric from the acceleration signal that is correlated to a frequency of oscillations of the acceleration signal, determine that the at least one frequency metric meets atrial tachyarrhythmia criteria; and detect an atrial tachyarrhythmia in response to at least the frequency metric meeting the atrial tachyarrhythmia criteria.

In another example, the disclosure provides a method including sensing an acceleration signal, determining at least one frequency metric from the acceleration signal that is correlated to a frequency of oscillations of the acceleration signal, determining that the at least one frequency metric meets atrial tachyarrhythmia criteria, and detecting an atrial tachyarrhythmia in response to at least the frequency metric meeting the atrial tachyarrhythmia criteria.

In another example, the disclosure provides a non-transitory, computer-readable storage medium storing a set of instructions which, when executed by a control circuit of a medical device, cause the medical device to sense an acceleration signal and determine at least one frequency metric from the acceleration signal that is correlated to a frequency of oscillations of the acceleration signal. The instructions further cause the medical device to determine that the at least one frequency metric meets atrial tachyarrhythmia criteria and detect an atrial tachyarrhythmia in response to at least the frequency metric meeting the atrial tachyarrhythmia criteria.

Further disclosed herein is the subject matter of the following clauses:
1. A medical device comprising: an accelerometer configured to sense an acceleration signal; a control circuit configured to receive the acceleration signal and determine from the acceleration signal at least one frequency metric that is correlated to a frequency of oscillations of the acceleration signal, determine that the at least one frequency metric meets atrial tachyarrhythmia criteria, and detect an atrial tachyarrhythmia in response to at least the frequency metric meeting the atrial tachyarrhythmia criteria.
2. The medical device of clause 1, wherein the control circuit is configured to:

determine the at least one frequency metric by:
performing a time-frequency transform of the acceleration signal;
determining a characteristic frequency of the acceleration signal based on the time-frequency transform; and

determine that the frequency metric meets atrial tachyarrhythmia criteria by determining that the characteristic frequency is greater than a frequency threshold.
3. The medical device of any of clauses 1-2, wherein the control circuit is configured to: determine the at least one frequency metric by setting a time interval and determining a count of acceleration signal oscillations during the time interval; and determine that the frequency metric meets atrial tachyarrhythmia criteria by determining that the count of acceleration signal oscillations is greater than a threshold value.
4. The medical device of any of clauses 1-3, wherein the control circuit is configured to determine the at least one frequency metric by setting a time interval and determining at least one of a low slope content, an integrated value, a median amplitude, a mean amplitude or a root mean square of the acceleration signal sensed over the time interval.
5. The medical device of any of clauses 1-4 comprising a cardiac electrical signal sensing circuit configured to sense a cardiac electrical signal and generate atrial sensed event signals in response to P-wave sensing threshold crossings by the cardiac electrical signal, wherein the control circuit is configured to: receive the atrial sensed event signals; determine that fast atrial rate criteria are met based on the atrial sensed event signals; and determine the at least on frequency metric from the acceleration signal in response to the fast atrial rate criteria being met.
6. The medical device of any of clauses 1-5 comprising a cardiac electrical signal sensing circuit configured to: sense a cardiac electrical signal; and generate atrial sensed event signals in response to P-wave sensing threshold crossings by the cardiac electrical signal, wherein the control circuit is configured to: receive the atrial sensed event signals generated by the cardiac electrical signal sensing circuit; determine a frequency metric threshold based on a frequency of the atrial sensed event signals; and determine that the atrial tachyarrhythmia criteria are met in response to the frequency metric being greater than the frequency metric threshold.
7. The medical device of any of clauses 1-6 comprising: a cardiac electrical signal sensing circuit configured to sense a cardiac electrical signal generate atrial sensed event signals in response to P-wave sensing threshold crossings by the cardiac electrical signal, wherein the control circuit is configured to: receive the atrial sensed event signals; disable the accelerometer in response to detecting the atrial tachyarrhythmia; determine that termination criteria are met based on the atrial sensed event signals; and detect termination of the atrial tachyarrhythmia episode in response to the termination criteria being met.
8. The medical device of any of clauses 1-7 comprising a temperature sensor configured to sense a temperature signal, wherein the control circuit is configured to: determine a patient physical activity metric based on the acceleration signal; determine a rate response pacing rate based on the patient physical activity metric; and responsive to determining that the atrial tachyarrhythmia criteria are met, adjust the rate response pacing rate based on the temperature signal.
9. The medical device of any of clauses 1-8, wherein the control circuit is configured to: determine the at least one frequency metric from the acceleration signal for each one of a plurality of time intervals; classify each one of the plurality of time intervals as one of an atrial tachyarrhythmia time interval or a non-atrial tachyarrhythmia time interval based on the frequency metric; and determine that the at least one frequency metric meets the atrial tachyarrhythmia criteria in response to determining that a threshold number of the plurality of time intervals are classified as atrial tachyarrhythmia time intervals.
10. The medical device of any of clauses 1-8, wherein the control circuit is configured to: determine a first frequency metric from the acceleration signal sensed during a first time interval having a first duration, the first frequency metric correlated to a frequency of oscillations of the acceleration signal; determine a second frequency metric from the acceleration signal sensed during a second time interval having a second duration different than the duration of the first time interval, the second frequency metric different than the first frequency metric; and determine that the first frequency metric and the second frequency metric meet the atrial tachyarrhythmia criteria.
11. The medical device of any of clauses 1-10 further comprising a pulse generator configured to generate pacing pulses according to a pacing therapy in response to the control circuit detecting that atrial tachyarrhythmia.
12. The medical device of any of clauses 1-11 further comprising a telemetry circuity configured to transmit an atrial tachyarrhythmia detection notification in response to the control circuit detecting the atrial tachyarrhythmia.
13. The medical device of any of clauses 1-12 further comprising a pulse generator and a housing enclosing the accelerometer, the control circuit, and the pulse generator, the housing comprising a pair of housing-based electrodes coupled to the pulse generator.
14. A method comprising: sensing an acceleration signal; determining at least one frequency metric from the acceleration signal that is correlated to a frequency of oscillations of the acceleration signal; determining that the at least one frequency metric meets atrial tachyarrhythmia criteria; and detecting an atrial tachyarrhythmia in response to at least the frequency metric meeting the atrial tachyarrhythmia criteria.
15. The method of clause 14, wherein: determining the at least one frequency metric comprises: performing a time-frequency transform of the acceleration signal; determining a characteristic frequency of the acceleration signal based on the time-frequency transform; and determining that the frequency metric meets atrial tachyarrhythmia criteria comprises determining that the characteristic frequency is greater than a frequency threshold.
16. The method of any of clauses 14-15, wherein determining the at least one frequency metric comprises setting a time interval and determining a count of acceleration signal oscillations during the time interval and wherein determining that the frequency metric meets atrial tachyarrhythmia criteria comprises determining that the count of acceleration signal oscillations is greater than a threshold value.
17. The method of any of clauses 14-16, wherein determining the at least one frequency metric comprises setting a time interval and determining at least one of a low slope content, an integrated value, a median amplitude, a mean amplitude or a root mean square of the acceleration signal sensed over the time interval.
18. The method of any of clauses 14-17 comprising sensing a cardiac electrical signal; generating atrial sensed event signals in response to P-wave sensing threshold crossings by the cardiac electrical signal; determining that fast atrial rate criteria are met based on the atrial sensed event signals; and determining the at least on frequency metric from the acceleration signal in response to the fast atrial rate criteria being met.
19. The method of any of clauses 14-18 comprising: sensing a cardiac electrical signal; generating atrial sensed event signals in response to P-wave sensing threshold crossings by the cardiac electrical signal; determining a frequency metric threshold based on a frequency of the atrial sensed event signals; and determining that the atrial tachyarrhythmia criteria are met in response to the frequency metric being greater than the frequency metric threshold.
20. The method of any of clauses 14-19 comprising: sensing a cardiac electrical signal; generating atrial sensed event signals in response to P-wave sensing threshold crossings by the cardiac electrical signal; disabling the accelerometer in response to detecting the atrial tachyarrhythmia; determining that termination criteria are met based on the atrial sensed event signals; and detecting termination of the atrial tachyarrhythmia episode in response to the termination criteria being met.
21. The method of any of clauses 14-20 comprising: determining a patient physical activity metric based on the acceleration signal; determining a rate response pacing rate based on the patient physical activity metric; sensing a temperature signal; and responsive to determining that the atrial tachyarrhythmia criteria are met, adjusting the rate response pacing rate based on the temperature signal.
22. The method of any of clauses 14-21, comprising: determining the at least one frequency metric from the acceleration signal for each one of a plurality of time intervals; classifying each one of the plurality of time intervals as one of an atrial tachyarrhythmia time interval or a non-atrial tachyarrhythmia time interval based on the frequency metric; and determining that the at least one frequency metric meets the atrial tachyarrhythmia criteria in response to determining that a threshold number of the plurality of time intervals are classified as atrial tachyarrhythmia time intervals.
23. The method of any of clauses 14-22, comprising: determining a first frequency metric from the acceleration signal sensed during a first time interval having a first duration, the first frequency metric correlated to a frequency of oscillations of the acceleration signal; determining a second frequency metric from the acceleration signal sensed during a second time interval having a second duration different than the first duration of the first time interval, the second frequency metric different than the first frequency metric; and determining that the first frequency metric and the second frequency metric meet the atrial tachyarrhythmia criteria.
24. The method of any of clauses 14-23 further comprising generating pacing pulses according to a pacing therapy in response to detecting that atrial tachyarrhythmia.
25. The method of any of clauses 14-24 further comprising transmitting an atrial tachyarrhythmia detection notification in response to detecting the atrial tachyarrhythmia.
26. A non-transitory, computer-readable storage medium storing a set of instructions which, when executed by a control circuit of a medical device, cause the medical device to: sense an acceleration signal; determine at least one frequency metric from the acceleration signal that is correlated to a frequency of oscillations of the acceleration signal; determine that the at least one frequency metric meets atrial tachyarrhythmia criteria; and detect an atrial tachyarrhythmia in response to at least the frequency metric meeting the atrial tachyarrhythmia criteria.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device (IMD) system that may be used to sense cardiac signals and perform atrial tachyarrhythmia and/or atrial fibrillation (AT/AF) detection.

FIG. 2 is a conceptual diagram of the transcatheter leadless pacemaker of FIG. 1 according to one example.

FIGS. 3A-3C are conceptual diagrams of a patient implanted with an IMD system that may include the atrial pacemaker of FIG. 1 according to another example.

FIG. 4 is a conceptual diagram of one configuration of an atrial pacemaker capable of sensing cardiac signals, detecting AT/AF and delivering pacing therapy.

FIG. 5 is a diagram of an electrocardiogram (ECG) signal during normal sinus rhythm and a corresponding acceleration signal and atrial electrogram (EGM) signal that may be sensed by the pacemaker of FIG. 1.

FIG. 6 is a diagram of an ECG signal during AF and a corresponding acceleration signal and atrial EGM signal that may be sensed by the pacemaker of FIG. 1.

FIG. 7 is a flow chart of a method for detecting AT/AF by a medical device according to some examples.

FIG. 8 is a flow chart of a method for detecting AT/AF by a medical device according to another example.

FIG. 9 is a diagram of an acceleration signal and an atrial EGM signal illustrating one method that may be executed by a control circuit of a medical device for determining a frequency metric from the acceleration signal.

FIG. 10 is a flow chart of a method for detecting and responding to AT/AF by a medical device according to another example.

### DETAILED DESCRIPTION

In general, this disclosure describes a medical device and techniques for detecting atrial tachyarrhythmia. The medical device is configured to sense an atrial acceleration signal from an accelerometer implanted in an atrial location, e.g., in or on an atrial chamber. According to the techniques disclosed herein, the medical device is configured to analyze the atrial acceleration signal for detecting an atrial tachyarrhythmia when the acceleration signal meets atrial tachyarrhythmia criteria. The atrial tachyarrhythmia criteria may be defined to discriminate between normal sinus tachycardia (NST) and non-sinus atrial tachycardia (AT) or atrial fibrillation (AF).

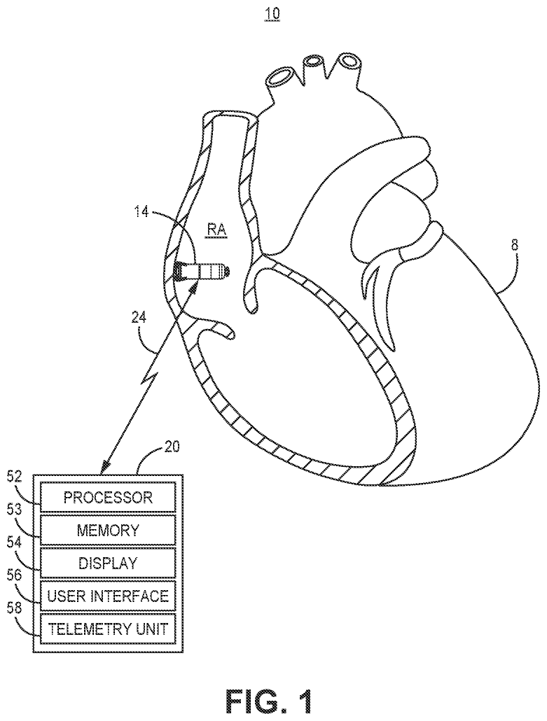

FIG. 1 is a conceptual diagram illustrating an implantable medical device (IMD) system 10 that may be used to sense cardiac signals and provide atrial tachyarrhythmia detection. IMD system 10 is shown including atrial pacemaker 14, shown implanted within the right atrium (RA). Pacemaker 14 may be a transcatheter leadless pacemaker which is implantable wholly within a heart chamber, e.g., wholly within the right atrium (RA) of heart 8 for sensing cardiac signals and delivering atrial pacing pulses from within the atrium. Pacemaker 14 may be implanted along the lateral endocardial wall as shown though other locations are possible within or on the RA, different than the location shown.

Pacemaker 14 includes housing-based electrodes for sensing cardiac electrical signals and delivering pacing pulses. Pacemaker 14 may include cardiac electrical signal sensing circuitry configured to sense atrial P-waves attendant to the depolarization of the atrial myocardium and a pulse generator for generating and delivering an atrial pacing pulse in the absence of a sensed atrial P-wave.

Pacemaker 14 includes an accelerometer enclosed within or on the housing of the pacemaker. The accelerometer is subjected to acceleration forces due to cardiac and blood motion. During normal sinus rhythm, the acceleration signal generated by the accelerometer may include signals that correspond to ventricular contraction and atrial contraction that occur at regular intervals and a frequency corresponding to the normal sinus rate. However, during AT or AF, the acceleration signal may include an increased frequency of oscillations that represent a different characteristic frequency than during sinus tachycardia or normal sinus rhythm. In particular, the frequency of oscillations during AT/AF may be approximately double the frequency of atrial electrical event signals in a sensed atrial electrical signal, e.g., an atrial EGM signal. As described below, pacemaker 14 may be configured to determine an acceleration signal frequency metric correlated to the frequency of oscillations of the acceleration signal for use in detecting AT/AF and discriminating AT/AF from sinus tachycardia. The frequency metric(s) determined by processing circuitry of pacemaker 14 from the acceleration signal may include one or more of a characteristic frequency of the acceleration signal, a count of oscillations of the acceleration signal over one or more atrial cycles or a predetermined time period, an integration, a mean or median amplitude, a slope content, a root mean square or other metric that is correlated (directly or inversely) to the frequency of oscillations of the acceleration signal.

The acceleration signal sensed by the accelerometer may include acceleration signals due to patient body motion, e.g., during physical activity, in addition to acceleration signals due to cardiac motion. The acceleration signal produced by the accelerometer may also be representative of patient physical activity, therefore, and used by processing circuitry included in the pacemaker 14 for determining a patient physical activity metric. The rate of cardiac pacing pulses generated and delivered by pacemaker 14 may be adjusted based on the patient physical activity metric determined from the accelerometer signal for providing rate response pacing in some examples.

Pacemaker 14 may include a second sensor for use in controlling the rate response pacing rate. The second sensor is a temperature sensor in some examples. During AT/AF, the increased frequency of oscillations of the accelerometer signal due to the AT or AF may contribute to an elevated patient physical activity metric determined from the acceleration signal. This increased contribution of cardiac motion to the acceleration signal during AT/AF may be a confounding factor in determining an actual patient physical activity metric that reflects the true physical activity level of the patient. AT/AF may contribute to the acceleration signal before and/or after the onset of increased patient physical activity. Intervals of non-sustained or intermittent AT/AF may occur, which may make the patient physical activity metric increase and decrease in a manner that is not representative of the true level of patient physical activity. The onset of AT/AF while the patient is at rest may cause an increase in the cardiac contribution to the accelerometer signal, potentially resulting in an increase in the patient physical activity metric determined from the acceleration signal and an increased rate response pacing rate delivered by the pacemaker 14. When the onset of AT/AF occurs before or during increased patient physical activity, the increased contribution of cardiac motion to the acceleration signal during patient physical activity may prevent or slow a decrease in the rate response pacing rate as patient physical activity declines or ceases.

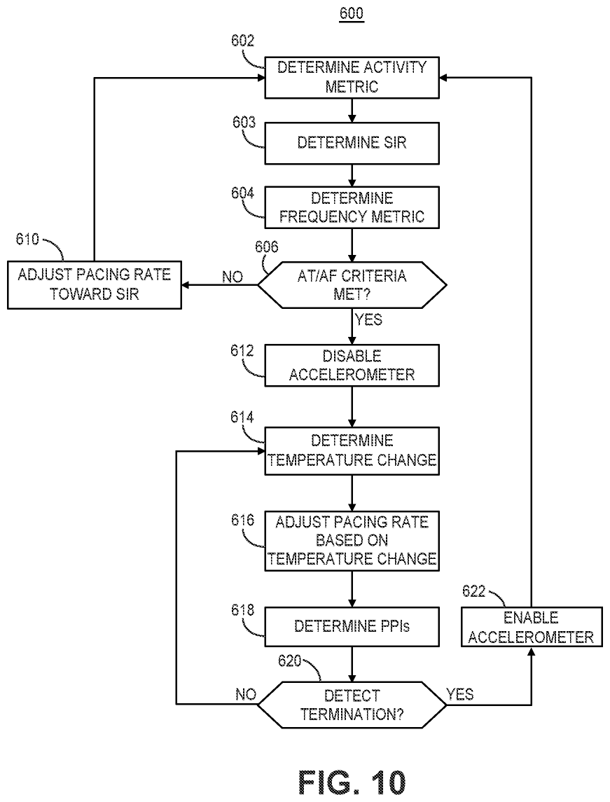

According to some examples, the second sensor, e.g., a temperature sensor, is included in pacemaker 14 to provide a second signal that is correlated to patient physical activity and metabolic need but is less sensitive to changes in cardiac motion. The second sensor signal may be used by pacemaker 14 to control rate response pacing in addition to the accelerometer signal. As described below, e.g., in conjunction with FIG. 10, when AT/AF is detected, the second sensor signal may be used to withhold an adjustment to the pacing rate based on the patient physical activity metric determined from the accelerometer signal. In other instances, the second sensor signal may be used directly to control the rate response pacing rate instead of the acceleration-based patient physical activity metric when AT/AF is detected.

Pacemaker 14 may be capable of bidirectional wireless communication with an external device 20 for programming sensing and pacing control parameters, which may include control parameters used for sensing the cardiac electrical signal, the acceleration signal and the temperature sensor signal (when included), control parameters used for detecting AT/AF and providing a response, and control parameters used for controlling atrial pacing. Aspects of external device 20 may generally correspond to the external programming/monitoring unit disclosed in U.S. Pat. No. 5,507,782 (Kieval, et al.), hereby incorporated herein by reference in its entirety. External device 20 is often referred to as a “programmer” because it is typically used by a physician, technician, nurse, clinician or other qualified user for programming operating parameters in an implantable medical device, e.g., pacemaker 14. External device 20 may be located in a clinic, hospital or other medical facility. External device 20 may alternatively be embodied as a home monitor or a handheld device that may be used in a medical facility, in the patient's home, or another location. Operating parameters, including sensing and therapy delivery control parameters, may be programmed into pacemaker 14 by a user interacting with external device 20.

External device 20 may include a processor 52, memory 53, display unit 54, user interface 56 and telemetry unit 58. Processor 52 controls external device operations and processes data and signals received from pacemaker 14. Display unit 54 may generate a display, which may include a graphical user interface, of data and information relating to pacemaker functions to a user for reviewing pacemaker operation and programmed parameters as well as cardiac electrical signals, accelerometer signals, second sensor signals or other physiological data that may be acquired by pacemaker 14 and transmitted to external device 20 during an interrogation session. For example, pacemaker 14 may generate an output for transmission to external device 20 relating to detected AT/AF episodes. Transmitted data may include an episode of a cardiac electrical signal and/or an acceleration signal produced by pacemaker sensing circuitry including markers indicating sensed cardiac event signals and AT/AF detection.

User interface 56 may include a mouse, touch screen, keypad or the like to enable a user to interact with external device 20 to initiate a telemetry session with pacemaker 14 for retrieving data from and/or transmitting data to the pacemaker 14, including programmable parameters for controlling AT/AF detection. Telemetry unit 58 includes a transceiver and antenna configured for bidirectional communication with a telemetry circuit included in pacemaker 14 and is configured to operate in conjunction with processor 52 for sending and receiving data relating to pacemaker functions via communication link 24. Telemetry unit 58 may establish a wireless bidirectional communication link 24 with pacemaker 14. Communication link 24 may be established using a radio frequency (RF) link such as BLUETOOTH®, Wi-Fi, Medical Implant Communication Service (MICS) or other communication bandwidth. In some examples, external device 20 may include a programming head that is placed proximate pacemaker 14 to establish and maintain a communication link 24. In other examples external device 20 and pacemaker 14 may be configured to communicate using a distance telemetry algorithm and circuitry that does not require the use of a programming head and does not require user intervention to maintain a communication link.

It is contemplated that external device 20 may be in wired or wireless connection to a communications network via a telemetry circuit that includes a transceiver and antenna or via a hardwired communication line for transferring data to a centralized database or computer to allow remote management of the patient. Remote patient management systems including a centralized patient database may be configured to utilize the presently disclosed techniques to enable a clinician to view data relating to sensing cardiac signals, AT/AF detection and pacing operations performed by pacemaker 14.

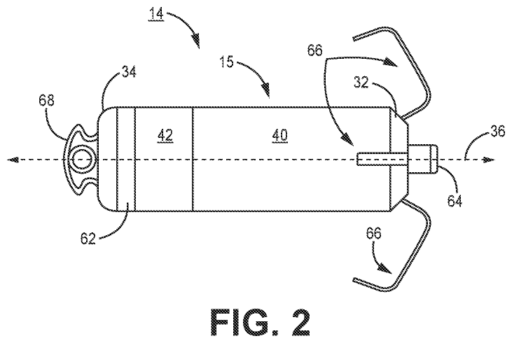

FIG. 2 is a conceptual diagram of the transcatheter leadless pacemaker 14 of FIG. 1 according to one example. Pacemaker 14 includes a housing 15 that may include a control electronics subassembly 40 and a battery subassembly 42, which provides power to the control electronics subassembly 40. Pacemaker 14 includes electrodes 62 and 64 spaced apart along the housing 15 of pacemaker 14 for sensing cardiac electrical signals and delivering pacing pulses. Electrode 64 is shown as a tip electrode extending from a distal end 32 of pacemaker 14, and electrode 62 is shown as a ring electrode circumscribing the lateral wall of housing 15, along a mid-portion of housing 15. In the example shown, electrode 62 is shown adjacent proximal end 34 of housing 15. Distal end 32 is referred to as “distal” in that it is expected to be the leading end of pacemaker 14 as pacemaker 14 is advanced through a delivery tool, such as a catheter, and placed against a targeted pacing site.

Electrodes 62 and 64 form an anode and cathode pair for bipolar cardiac pacing and sensing. In alternative embodiments, pacemaker 14 may include two or more ring electrodes, two tip electrodes, and/or other types of electrodes exposed along pacemaker housing 15 for delivering electrical stimulation to heart 8 and sensing cardiac electrical signals. Electrodes 62 and 64 may be, without limitation, titanium, platinum, iridium or alloys thereof and may include a low polarizing coating, such as titanium nitride, iridium oxide, ruthenium oxide, platinum black, among others. Electrodes 62 and 64 may be positioned at locations along pacemaker 14 other than the locations shown and may include ring, button, hemispherical, hook, helical or other types of electrodes.

Housing 15 is formed from a biocompatible material, such as a stainless steel or titanium alloy. In some examples, the housing 15 may include an insulating coating. Examples of insulating coatings include parylene, urethane, PEEK, or polyimide, among others. The entirety of the housing 15 may be insulated, but only electrodes 62 and 64 uninsulated. Electrode 64 may serve as a cathode electrode and be coupled to internal circuitry, e.g., a pacing pulse generator and cardiac electrical signal sensing circuitry, enclosed by housing 15 via an electrical feedthrough crossing housing 15. Electrode 62 may be formed as a conductive portion of housing 15 defining a ring electrode that is electrically isolated from the other portions of the housing 15 as generally shown in FIG. 2. In other examples, the entire periphery of the housing 15 may function as an electrode that is electrically isolated from tip electrode 64, instead of providing a localized ring electrode such as electrode 62. Electrode 62 formed along an electrically conductive portion of housing 15 serves as a return anode during pacing and sensing.

Control electronics subassembly 40 houses the electronics for sensing cardiac signals, detecting arrhythmias, producing pacing pulses and controlling therapy delivery and other functions of pacemaker 14 as described herein. A motion sensor implemented as an accelerometer may be enclosed within housing 15 in some examples. The accelerometer provides a signal to a processor included in control electronics subassembly 52 for signal processing and analysis for detecting AT/AF and may be used determining a patient physical activity metric for use in controlling rate response cardiac pacing.

The accelerometer may be a multi-axis or multi-dimensional accelerometer where each axis of the accelerometer generates an acceleration signal in a different dimension. In some examples, the accelerometer is a three-dimensional accelerometer having one “longitudinal” axis that is parallel to or aligned with the longitudinal axis 36 of pacemaker 14 and two orthogonal axes that extend in radial directions relative to the longitudinal axis 36. Practice of the techniques disclosed herein, however, are not limited to a particular orientation of the accelerometer within or along housing 15 or a particular number of axes. In other examples, a one-dimensional accelerometer may be used to obtain an acceleration signal which may be analyzed for detecting AT/AF and, in some examples, determine a patient physical activity metric. In still other examples, a two dimensional accelerometer or other multi-dimensional accelerometer may be used. Each axis of a single or multi-dimensional accelerometer may be defined by a piezoelectric element, micro-electrical mechanical system (MEMS) device or other sensor element capable of producing an electrical signal in response to changes in acceleration imparted on the sensor element, e.g., by converting the acceleration to a force or displacement that is converted to the electrical signal. In a multi-dimensional accelerometer, the sensor elements may be arranged orthogonally with each sensor element axis orthogonal relative to the other sensor element axes. Orthogonal arrangement of the elements of a multi-axis accelerometer, however, is not necessarily required.

Each sensor element or axis may produce an acceleration signal corresponding to a vector aligned with the axis of the sensor element. A vector signal of a multi-dimensional accelerometer (also referred to herein as a “multi-axis” accelerometer) for use in monitoring acceleration signals for AT/AF detection and for monitoring patient physical activity may be selected as a single axis signal or a combination of two or more axis signals. For example, one, two or all three axis signals produced by a three-dimensional accelerometer may be selected for processing and analysis by a control circuit of pacemaker 14 for use in determining a frequency metric and detecting AT/AF based on the frequency metric. In a three-dimensional accelerometer, having one axis aligned with longitudinal axis 36 and two axes aligned orthogonally in two radial directions, one of the axis signals may be selected as a default axis for obtaining an acceleration signal for determining a frequency metric that is correlated to oscillations of the acceleration signal that occur due to atrial motion. The axis signal or combination of axis signals used for determining a frequency metric, however, may be selectable and may be programmable by a user. The axis signal or combination of axis signals analyzed for detecting AT/AF may be the same or different than the axis signal or combination of axis signals used for determining a patient physical activity metric for controlling rate response pacing. In some examples, the vector selection techniques for monitoring patient physical activity generally disclosed in U.S. Pat. No. 10,512,424 (Demmer, et al.) may be implemented in conjunction with the techniques disclosed herein. The '424 reference is incorporated herein by reference in its entirety.

As described above, pacemaker 14 may include a second sensor on or enclosed by housing 15 for producing a signal correlated to metabolic demand for use in controlling rate response pacing. For instance, pacemaker 14 may include a temperature sensor enclosed by housing 15 as a second sensor for controlling rate response. When pacemaker 14 is implanted in or on the patient's heart, the accelerometer is subjected to acceleration forces due to cardiac motion as well as patient body motion. During AT/AF, acceleration signals due to atrial motion may contribute to a patient physical activity metric, which could result in pacemaker 14 increasing the pacing rate to provide rate response pacing when the patient may actually not require an increased pacing rate. The second sensor, such as a temperature sensor, may be less sensitive or insensitive to atrial motion during AT/AF and provide a better indication of patient physical activity and metabolic demand than the accelerometer signal during AT/AF. Accordingly, pacemaker 14 may include a temperature sensor in addition to the accelerometer and process both signals for determining an appropriate pacing rate response.

Pacemaker 14 may include features for facilitating deployment and fixation of pacemaker 14 at an implant site. For example, pacemaker 14 may include a set of fixation tines 66 to secure pacemaker 14 to patient tissue, e.g., by actively engaging with the atrial pectinate muscle or atrial endocardial tissue. Fixation tines 66 are configured to anchor pacemaker 14 to position electrode 64 in operative proximity to a targeted tissue for delivering therapeutic electrical stimulation pulses. Numerous types of active and/or passive fixation members may be employed for anchoring or stabilizing pacemaker 14 in an implant position.

Pacemaker 14 may optionally include a delivery tool interface 68. Delivery tool interface 68 may be located at the proximal end 34 of pacemaker 14 and is configured to connect to a delivery device, such as a catheter, used to position pacemaker 14 at an implant site during an implantation procedure, for example within or on an atrial chamber.

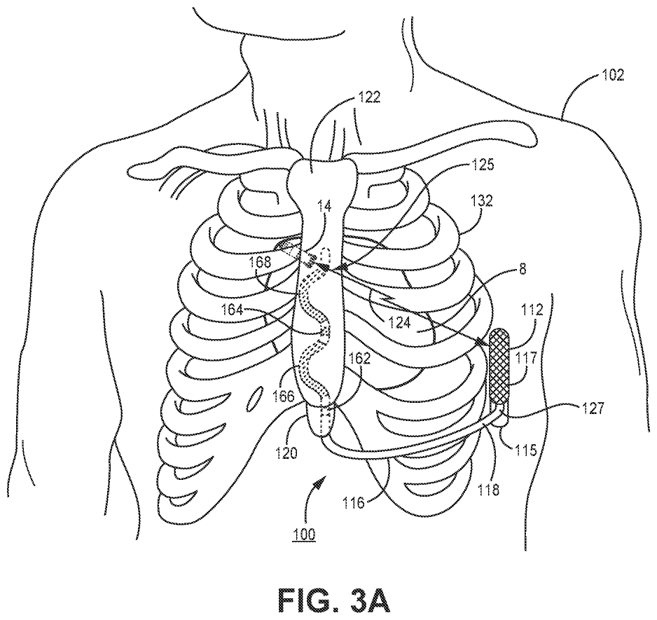

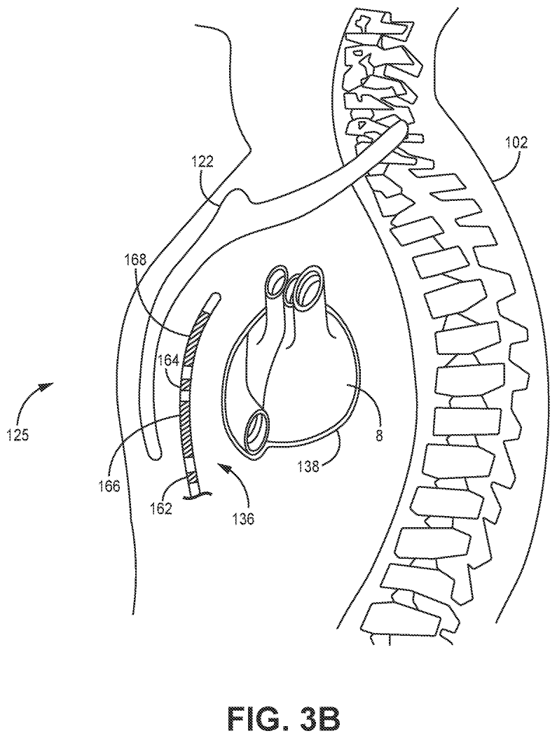

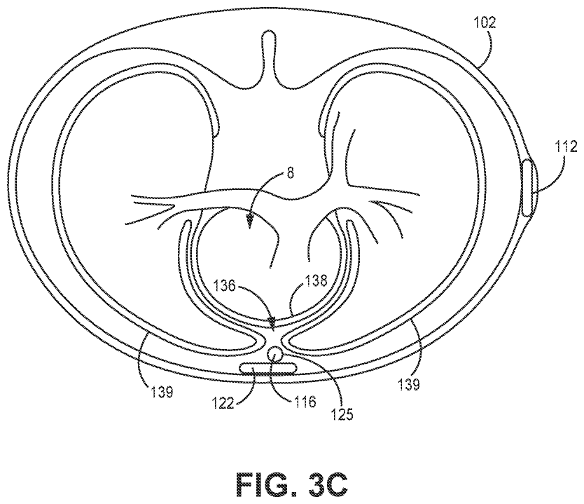

FIGS. 3A-3C are conceptual diagrams of a patient 102 implanted with an IMD system 100 that may include atrial pacemaker 14 according to another example. FIG. 3A is a front view of patient 102 implanted with IMD system 100. FIG. 3B is a side view of patient 102 implanted with IMD system 100. FIG. 3C is a transverse view of patient 102 implanted with IMD system 100. In this example, IMD system 100 includes an implantable cardioverter defibrillator (ICD) 112 connected to an extra-cardiovascular electrical stimulation and sensing lead 116. In the implant configuration shown, lead 116 is implanted at least partially underneath sternum 122 of patient 102. Lead 116 extends subcutaneously or submuscularly from ICD 112 toward xiphoid process 120 and at a location near xiphoid process 120 bends or turns and extends superiorly within anterior mediastinum 136 (see FIGS. 3B and 3C) in a substernal position. The path of extra-cardiovascular lead 116 may depend on the location of ICD 112, the arrangement and position of electrodes carried by the lead body 118, and/or other factors. The techniques disclosed herein are not limited to a particular path of lead 116 or final locations of electrodes carried by lead body 118.

Anterior mediastinum 136 may be viewed as being bounded laterally by pleurae 139, posteriorly by pericardium 138, and anteriorly by sternum 122. The distal portion 125 of lead 116 may extend along the posterior side of sternum 122 substantially within the loose connective tissue and/or substernal musculature of anterior mediastinum 136. A lead implanted such that the distal portion 125 is substantially within anterior mediastinum 136, or within a pleural cavity or more generally within the thoracic cavity, may be referred to as a “substernal lead.”

In the example illustrated in FIGS. 3A-3C, the distal portion 125 of lead 116 is located substantially centered under sternum 122. In other instances, however, lead 116 may be implanted such that the distal portion 125 may be offset laterally from the center of sternum 122. In some instances, lead 116 may extend laterally such that distal portion 125 is underneath/below the ribcage 132 in addition to or instead of sternum 122. In other examples, the distal portion 125 of lead 116 may be implanted in other extra-cardiac, intra-thoracic locations, including the pleural cavity or around the perimeter of and adjacent to or within the pericardium 138 of heart 8.

ICD 112 includes a housing 115 that forms a hermetic seal that protects internal components of ICD 112. The housing 115 of ICD 112 may be formed of a conductive material, such as titanium or titanium alloy. The housing 115 may function as an electrode (sometimes referred to as a “can” electrode). Housing 115 may be used as an active can electrode for use in delivering CV/DF shocks or other high voltage pulses delivered using a high voltage therapy circuit. In other examples, housing 115 may be available for use in delivering unipolar, low voltage cardiac pacing pulses and/or for sensing cardiac electrical signals in combination with electrodes carried by lead 116. In other instances, the housing 115 of ICD 112 may include a plurality of electrodes on an outer portion of the housing. The outer portion(s) of the housing 115 functioning as an electrode(s) may be coated with a material, such as titanium nitride, e.g., for reducing post-stimulation polarization artifact.

ICD 112 includes a connector assembly 117 (also referred to as a connector block or header) that includes electrical feedthroughs crossing housing 115 to provide electrical connections between conductors extending within the lead body 118 of lead 116 and electronic components included within the housing 115 of ICD 112. Housing 115 may house one or more processors, memories, transceivers, cardiac electrical signal sensing circuitry, therapy delivery circuitry, power sources and other components for sensing cardiac electrical signals, detecting a heart rhythm, and controlling and delivering electrical stimulation pulses to treat an abnormal heart rhythm.

Lead 116 includes an elongated lead body 118 having a proximal end 127 that includes a lead connector (not shown) configured to be connected to ICD connector assembly 117 and a distal portion 125 that includes one or more electrodes. In the example illustrated in FIGS. 3A-3C, the distal portion 125 of lead body 118 includes defibrillation electrodes 166 and 168 and pace/sense electrodes 162 and 164. In some cases, defibrillation electrodes 166 and 168 may together form a defibrillation electrode in that they may be configured to be activated concurrently. Alternatively, defibrillation electrodes 166 and 168 may form separate defibrillation electrodes in which case each of the electrodes 166 and 168 may be activated independently.

Electrodes 166 and 168 (and in some examples housing 115) are referred to herein as defibrillation electrodes because they may be utilized, individually or collectively, for delivering high voltage stimulation therapy (e.g., cardioversion or defibrillation shocks). Electrodes 166 and 168 may be elongated coil electrodes and generally have a relatively high surface area for delivering high voltage electrical stimulation pulses compared to pacing and sensing electrodes 162 and 164. However, electrodes 166 and 168 and housing 115 may also be utilized to provide pacing functionality, sensing functionality, or both pacing and sensing functionality in addition to or instead of high voltage stimulation therapy. In this sense, the use of the term “defibrillation electrode” herein should not be considered as limiting the electrodes 166 and 168 for use in only high voltage cardioversion/defibrillation shock therapy applications. For example, either of electrodes 166 and 168 may be used as a sensing electrode in a sensing vector for sensing cardiac electrical signals and determining a need for an electrical stimulation therapy.

Electrodes 162 and 164 are relatively smaller surface area electrodes which are available for use in sensing electrode vectors for sensing cardiac electrical signals and may be used for delivering relatively low voltage pacing pulses in some configurations, e.g., for delivering rate response pacing pulses. Electrodes 162 and 164 are referred to as pace/sense electrodes because they are generally configured for use in low voltage applications, e.g., used as either a cathode or anode for delivery of pacing pulses and/or sensing of cardiac electrical signals, as opposed to delivering high voltage CV/DF shocks. In some instances, electrodes 162 and 164 may provide only pacing functionality, only sensing functionality or both.

ICD 112 may obtain cardiac electrical signals corresponding to electrical activity of heart 8 via a combination of sensing electrode vectors that include combinations of electrodes 162, 164, 166 and/or 168. In some examples, housing 115 of ICD 112 is used in combination with one or more of electrodes 162, 164, 166 and/or 168 in a sensing electrode vector. In the example illustrated in FIGS. 3A-3C, electrode 162 is located proximal to defibrillation electrode 166, and electrode 164 is located between defibrillation electrodes 166 and 168. One, two or more pace/sense electrodes (or none) may be carried by lead body 118 and may be positioned at different locations along distal lead portion 125 than the locations shown. Electrodes 162 and 164 are illustrated as ring electrodes; however, electrodes 162 and 164 may comprise any of a number of different types of electrodes, including ring electrodes, short coil electrodes, hemispherical electrodes, directional electrodes, segmented electrodes, or the like.

Electrical conductors (not illustrated) extend through one or more lumens of the elongated lead body 118 of lead 116 from the lead connector at the proximal lead end 127 to electrodes 162, 164, 166, 168. Elongated electrical conductors contained within the lead body 118, which may be separate respective insulated conductors within the lead body 118, are each electrically coupled with respective defibrillation electrodes 166 and 168 and pace/sense electrodes 162 and 164. The respective conductors electrically couple the electrodes 162, 164, 166, 168 to circuitry, such as a therapy delivery circuit and/or a sensing circuit, of ICD 112 via connections in the connector assembly 117, including associated electrical feedthroughs crossing housing 115. The electrical conductors transmit therapy from a therapy delivery circuit within ICD 112 to one or more of defibrillation electrodes 166 and 168 and/or pace/sense electrodes 162 and 164 and transmit cardiac electrical signals from the patient's heart 8 from one or more of electrodes 162, 164, 166, 168 to the sensing circuit within ICD 112.

The lead body 118 of lead 116 may be formed from a non-conductive material, including silicone, polyurethane, fluoropolymers, mixtures thereof, and/or other appropriate materials, and shaped to form one or more lumens within which the one or more conductors extend. Lead body 118 may be tubular or cylindrical in shape. In other examples, the distal portion 125 (or all of) the elongated lead body 118 may have a flat, ribbon or paddle shape. Lead body 118 may be formed having a preformed distal portion 125 that is generally straight, curving, bending, serpentine, undulating or zig-zagging. In the example shown, lead body 118 includes a curving distal portion 125 having two “C” shaped curves, which together may resemble the Greek letter epsilon, “c.” The techniques disclosed herein are not limited to any particular lead body design, however. In other examples, lead body 118 is a flexible elongated lead body without any pre-formed shape, bends or curves.

ICD 112 analyzes the cardiac electrical signals received from one or more sensing electrode vectors to monitor for abnormal rhythms, such as bradycardia, ventricular tachycardia (VT) or ventricular fibrillation (VF). ICD 112 may analyze the heart rate and morphology of the cardiac electrical signals to monitor for tachyarrhythmia in accordance with any of a number of tachyarrhythmia detection techniques. ICD 112 generates and delivers electrical stimulation therapy in response to detecting a tachyarrhythmia (e.g., VT or VF) using a therapy delivery electrode vector which may be selected from any of the available electrodes 24, 26, 28 30 and/or housing 15. ICD 112 may deliver ATP in response to VT detection and in some cases may deliver ATP prior to a CV/DF shock or during high voltage capacitor charging in an attempt to avert the need for delivering a CV/DF shock. If ATP does not successfully terminate VT or when VF is detected, ICD 112 may deliver one or more CV/DF shocks via one or both of defibrillation electrodes 166 and 168 and/or housing 115. ICD 112 may generate and deliver other types of electrical stimulation pulses such as post-shock pacing pulses, asystole pacing pulses, or bradycardia pacing pulses using a pacing electrode vector that includes one or more of the electrodes 162, 164, 166, 168 and the housing 115 of ICD 112.

ICD 112 is shown implanted subcutaneously on the left side of patient 102 along the ribcage 132. ICD 112 may, in some instances, be implanted between the left posterior axillary line and the left anterior axillary line of patient 102. ICD 112 may, however, be implanted at other subcutaneous or submuscular locations in patient 102. For example, ICD 112 may be implanted in a subcutaneous pocket in the pectoral region. In this case, lead 116 may extend subcutaneously or submuscularly from ICD 112 toward the manubrium of sternum 22 and bend or turn and extend inferiorly from the manubrium to the desired location subcutaneously, submuscularly, substernally, over or beneath the ribcage 132. In yet another example, ICD 112 may be placed abdominally.

Lead 116 is shown in this example as an extra-cardiovascular lead implanted in a substernal location. In other examples, lead 116 may be implanted outside the ribcage and sternum, e.g., in a suprasternal location or adjacent sternum 122, over ribcage 132. While ICD 112 is shown coupled to a non-transvenous lead 116 positioned in an extra-cardiovascular location, in other examples ICD 112 may be coupled to a transvenous lead that positions electrodes within a blood vessel but may remain outside the heart in an extra-cardiac location. For example, a transvenous medical lead may be advanced along a venous pathway to position electrodes within the internal thoracic vein (ITV), an intercostal vein, the superior epigastric vein, or the azygos, hemiazygos, or accessory hemiazygos veins, as examples.

IMD system 100 is shown including pacemaker 14, shown conceptually as being implanted within the right atrium in FIG. 3A. ICD 112 and pacemaker 14 may be configured for bi-directional communication via telemetry link 124. Pacemaker 14 may be configured to transmit an AT/AF detection signal for receipt by ICD 112. ICD 112 may be configured to respond to a transmitted AT/AF detection signal by withholding a VT/VF detection and/or withhold a VT/VF therapy, e.g., a shock therapy or anti-tachycardia pacing. In other examples, ICD 112 may deliver a cardioversion shock in response to receiving an AT/AF notification signal transmitted by pacemaker 14 indicating a sustained AT/AF episode is being detected. ICD 112 may deliver cardioversion therapy in an attempt to terminate the AT/AF episode.

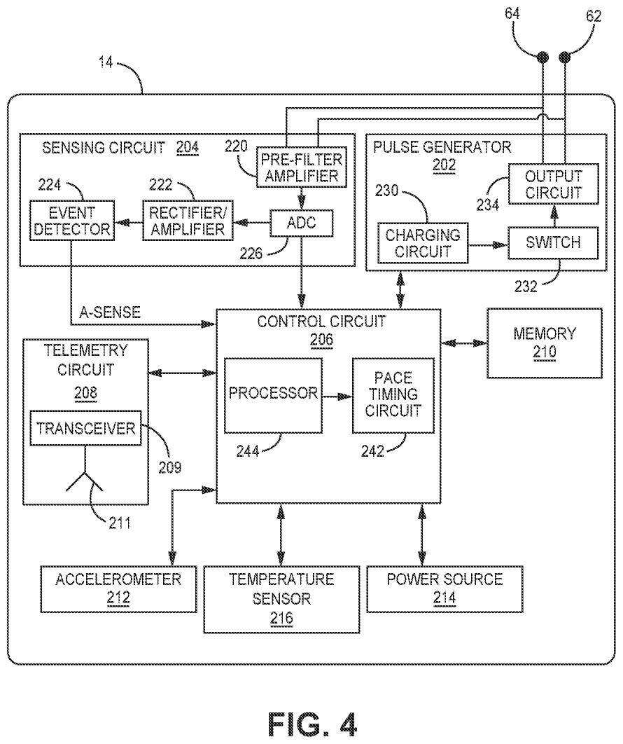

FIG. 4 is a conceptual diagram of an example configuration of atrial pacemaker 14 configured to sense cardiac signals, detect AT/AF and deliver pacing therapy according to one example. Pacemaker 14 includes a pulse generator 202, a cardiac electrical signal sensing circuit 204, a control circuit 206, memory 210, telemetry circuit 208, accelerometer 212, a power source 214, and in some examples a temperature sensor 216. The various circuits represented in FIG. 4 may be combined on one or more integrated circuit boards which include a specific integrated circuit (ASIC), an electronic circuit, a processor (shared, dedicated, or group) and memory that execute one or more software or firmware programs, a combinational logic circuit, state machine or other suitable components that provide the described functionality.

Sensing circuit 204 is configured to receive at least one cardiac electrical signal via electrodes coupled to pacemaker 14, e.g., electrodes 62 and 64. The cardiac electrical signal from electrodes 62 and 64 is received by a pre-filter and amplifier circuit 220. Pre-filter and amplifier circuit 220 may include a high pass filter to remove DC offset, e.g., a 2.5 to 5 Hz high pass filter, or a wideband filter having a bandpass of 2.5 Hz to 100 Hz or narrower to remove DC offset and high frequency noise. Pre-filter and amplifier circuit 220 may further include an amplifier to amplify the “raw” cardiac electrical signal passed to analog-to-digital converter (ADC) 226. ADC 226 may pass a multi-bit, digital electrogram (EGM) signal to control circuit 206 for use by control circuit 206 in identifying cardiac electrical events (e.g., P-waves attendant to atrial depolarizations) or performing morphology analysis for detecting various atrial arrhythmias. The digital signal from ADC 226 may be passed to rectifier and amplifier circuit 222, which may include a rectifier, narrow bandpass filter, and amplifier for passing the atrial electrical signal to cardiac event detector 224.

Cardiac event detector 224 may include a sense amplifier, comparator or other detection circuitry that compares the incoming rectified, cardiac electrical signal to a cardiac event sensing threshold, which may be an auto-adjusting threshold. For example, when the incoming signal crosses a P-wave sensing threshold, the cardiac event detector 224 generates an atrial sensed event signal (A-sense) that is passed to control circuit 206. In other examples, cardiac event detector 224 may receive the digital output of ADC 226 for sensing P-waves by a comparator, waveform morphology analysis of the digital EGM signal or other P-wave sensing techniques.

Processor 244 may provide sensing control signals to sensing circuit 204, e.g., P-wave sensing threshold control parameters such as sensitivity and various blanking and refractory intervals applied to the atrial electrical signal for controlling P-wave sensing. Atrial sensed event signals passed from cardiac event detector 224 to control circuit 206 may be used for scheduling atrial pacing pulses by pace timing circuit 242.

Accelerometer 212 may include piezoelectric sensors or MEMS devices for sensing an atrial acceleration signal. Accelerometer 212 may be a single axis accelerometer or a multi-axis accelerometer, e.g., a two-dimensional or three-dimensional accelerometer, with each axis providing an axis signal that may be analyzed individually or in combination for sensing acceleration signals. Accelerometer 212 produces an electrical signal correlated to motion or vibration of accelerometer 212 (and pacemaker 14), e.g., when subjected to flowing blood, cardiac motion and patient body motion.

One example of an accelerometer for use in implantable medical devices that may be implemented in conjunction with the techniques disclosed herein is generally disclosed in U.S. Pat. No. 5,885,471 (Ruben, et al.), incorporated herein by reference in its entirety. An implantable medical device arrangement including a piezoelectric accelerometer is disclosed, for example, in U.S. Pat. No. 4,485,813 (Anderson, et al.) and U.S. Pat. No. 5,052,388 (Sivula, et al.), both of which patents are hereby incorporated by reference herein in their entirety. Examples of three-dimensional accelerometers that may be implemented in pacemaker 14 and used for sensing acceleration signals are generally described in U.S. Pat. No. 5,593,431 (Sheldon) and U.S. Pat. No. 6,044,297 (Sheldon), both of which are incorporated herein by reference in their entirety. Other accelerometer configurations may be used for producing an electrical signal that is correlated to motion or acceleration forces imparted on pacemaker 14, which may be due to cardiac motion and patient body motion.

The accelerometer 212 may include one or more filter, amplifier, rectifier, analog-to-digital converter (ADC) and/or other components for producing an acceleration signal that may be passed to control circuit 206 for use in determining a frequency metric correlated to the frequency of oscillations of the acceleration signal, which may be representative of the atrial rhythm, e.g., a sinus rhythm vs. a non-sinus AT/AF. Control circuit 206 may additionally determine a patient physical activity metric for controlling rate response pacing from the acceleration signal received from accelerometer 212.

In various examples, the acceleration signal received from accelerometer 212 may be filtered by a high pass filter, e.g., a 10 Hz high pass filter, or a bandpass filter, e.g., a 10 Hz to 30 Hz bandpass filter. The filtered signal may be digitized by an ADC and optionally rectified for use by control circuit 240 for determining a frequency metric that may be used to discriminate between an atrial sinus rhythm and AT/AF. The high pass filter may be raised (e.g., to 15 Hz) if needed to detect acceleration signal oscillations that have higher frequency content during AT/AF. In some examples, high pass filtering is performed with no low pass filtering. In other examples, each accelerometer axis signal is filtered by a low pass filter, e.g., a 30 Hz low pass filter, with or without high pass filtering.

Additionally, a vector signal produced by an individual axis or combination of two or more axes of a multi-axis accelerometer may be filtered by a band pass or low pass filter, e.g., a 1-10 Hz bandpass filter or a 10 Hz low pass filter, digitized by an ADC and rectified for use by processor 244 of control circuit 206 for determining a patient physical activity metric. Various activity metrics may be derived from the accelerometer signal by control circuit 206 that are correlated to patient physical activity. In the illustrative examples presented herein, the accelerometer-based activity metric derived from the accelerometer signal is obtained by integrating the absolute value of a selected accelerometer vector signal over a predetermined time duration (such as 2 seconds). For example, the selected accelerometer axis signal may be filtered by a 1-10 Hz bandpass filter, rectified and sampled at 128 Hz in one example. The amplitude of the sampled data points over a two-second interval may be summed to obtain the activity metric. This activity metric may be referred to as an “activity count” and is correlated to the acceleration due to patient body motion imparted on the pacemaker 14 during the predetermined time interval. The 2-second (or other time interval) activity counts may be used by control circuit 206 for determining a sensor indicated pacing rate (SIR) for use in controlling rate response pacing. In other examples, the activity count may be further processed, e.g., the 2-second interval activity counts may be averaged or summed over multiple intervals, to determine a patient physical activity metric for use in controlling rate response pacing.

Example techniques for determining activity counts are generally disclosed in commonly-assigned U.S. Pat. No. 6,449,508 (Sheldon, et al.), incorporated herein by reference in its entirety. In other examples, an activity count may be determined as the number of sample points of the accelerometer signal that are greater than a predetermined threshold during a predetermined time interval. The techniques disclosed herein are not limited to a particular method for determining a patient physical activity metric from the accelerometer signal and other methods may be used to determine the accelerometer-based patient physical activity metric. Furthermore, the techniques for detecting AT/AF based at least in part on an acceleration signal from accelerometer 212 are not required to be implemented in a pacemaker configured to provide rate response pacing based on patient physical activity metrics determined from an acceleration signal.

In some examples, pacemaker 14 includes temperature sensor 216 as a second sensor representative of metabolic demand for use in controlling rate response pacing. Temperature sensor 216 may include one or more temperature sensors, e.g., thermocouples or thermistors, configured to produce a signal correlated to temperature surrounding housing 15, e.g., correlated to venous blood within the right atrium. Temperature sensor 216 may be disposed internally within the housing 15 of pacemaker 14, contacting the housing, formed as a part of the housing, or disposed external of the housing 15. As described herein, temperature sensor 216 may be used to measure absolute or relative changes in temperature of blood/tissue surrounding and/or contacting the housing 15 of pacemaker 14.

Processor 244 may receive a temperature signal from temperature sensor 216 to detect changes in temperature, e.g., in the blood or core body temperature, that occur with changing metabolic demand during patient physical activity. Although a single temperature sensor may be adequate, multiple temperature sensors may be included in temperature sensor 216 to generate a more accurate temperature profile or average temperature signal. Control circuit 206 may continually sample the temperature signal at a desired sampling rate from temperature sensor 216. However, control circuit 206 may conserve energy from power source 214 by only sampling temperature when AT/AF is being detected by control circuit 206, when the acceleration signal may be unreliable for determining a patient physical activity metric for controlling rate response pacing. In other examples, control circuit 206 may increase the rate of sampling a temperature signal in response to AT/AF being detected.

While a second sensor included in pacemaker 14 for use in controlling rate response pacing during AT/AF, it is contemplated that other types of sensors that are less sensitive to cardiac motion than accelerometer 212 and still produce a signal that is correlated to patient physical activity or metabolic demand may be included in pacemaker 14 to provide a second signal for use in controlling rate response pacing during AT/AF. Another example of a second sensor that may be included in pacemaker 14 is a blood oxygen saturation sensor for detecting changes in venous oxygen saturation within the RA for instance, which may occur with changes in patient physical activity.

Control circuit 206 includes pace timing circuit 242 and processor 244. Control circuit 206 may receive atrial sensed event signals and/or digital cardiac electrical signals from sensing circuit 204 for use in detecting and confirming P-waves and detecting AT/AF and controlling atrial pacing. For example, atrial sensed event signals may be passed to pace timing circuit 242 for starting a new atrial pacing escape interval for use in controlling the timing of pacing pulses delivered by pulse generator 202. Processor 244 may include one or more clocks for generating clock signals that are used by pace timing circuit 242 to time out a pacing escape interval, e.g., a permanent lower rate pacing interval for treating bradycardia or a temporary lower rate interval for providing rate response pacing. The pacing escape interval may be restarted by pace timing circuit 242 in response to each cardiac electrical event, e.g., upon receipt of each atrial sensed event signal from event detector 224 or upon delivery of each atrial pacing pulse by pulse generator 202.

When an atrial sensed event signal is received by control circuit 206 before the pacing escape interval expires, pace timing circuit 242 may pass the time elapsed of the pacing escape interval to processor 244 as the atrial event interval, e.g., a PP interval (PPI), between two consecutively sensed atrial events (or between an atrial pacing pulse and a subsequently sensed atrial event signal). When an atrial sensed event signal is not received by control circuit 206 before expiration of the pacing escape interval, pulse generator 202 generates an atrial pacing pulse in response to the pacing escape interval expiration. The pacing escape interval may be adjusted according to a rate response pacing rate that is set by control circuit 206 based on the accelerometer signal and/or the temperature signal according some examples.

Pulse generator 202 generates electrical pacing pulses upon expiration of a pacing escape interval set by pace timing circuit 242. The pacing pulses are delivered to the patient's heart via cathode electrode 64 and return anode electrode 62. Processor 244 may retrieve programmable pacing control parameters from memory 210, such as pacing pulse amplitude and pacing pulse width, which are passed to pulse generator 202 for controlling pacing pulse delivery. Pulse generator 202 may include charging circuit 230, switching circuit 232 and an output circuit 234. Charging circuit 230 is configured to receive current from power source 214 and may include a holding capacitor that may be charged to a pacing pulse amplitude under the control of a voltage regulator included in charging circuit 230. The pacing pulse amplitude may be set based on a control signal from control circuit 206. Switching circuit 232 may control when the holding capacitor of charging circuit 230 is coupled to the output circuit 234 for delivering the pacing pulse. For example, switching circuit 232 may include a switch that is activated by a timing signal received from pace timing circuit 242 upon expiration of a pacing escape interval and kept closed for a programmed pacing pulse width to enable discharging of the holding capacitor of charging circuit 230. The holding capacitor, previously charged to the pacing pulse voltage amplitude, is discharged across electrodes 62 and 64 (or other selected pacing electrode vector when available) through the output capacitor of output circuit 234 for the programmed pacing pulse duration.

Processor 244 may receive PPIs from pace timing circuit 242 for detecting PPIs meeting AT/AF detection interval criteria. For example, AT and/or AF detection interval zones may be defined which are compared to a PPI by processor 244. When a PPI falls in an AT or AF detection interval zone, a counter may be increased to count the number of AT/AF intervals. Separate and/or combined AT and AF interval counters may be provided. In some examples a counter may be configured to count the number of consecutive PPIs falling into an AT/AF interval zone. The counter may be reset to zero when a PPI is longer than an AT/AF detection interval. In other examples, a counter may be configured as an X of Y counter for counting how many PPIs fall into an AT/AF detection interval zone out of a predetermined number of most recent PPIs. When at least X of Y AT/AF intervals are detected, an AT/AF episode may be suspected based on the cardiac electrical signal.

Additionally or alternatively, processor 244 may analyze an atrial EGM signal received from ADC 226 for performing morphology analysis of the atrial electrical signal for detecting AT/AF morphology in support of AT/AF detection criteria being met. The morphology of an unknown atrial sensed event may be compared to a known, sinus P-wave template, for example, for classifying the unknown atrial sensed event as a sinus P-wave or a non-sinus event (AT/AF event), which may be counted toward AT/AF detection.

In some examples, when a count of AT/AF intervals and/or AT/AF events reaches a first threshold value, processor 244 may analyze a signal from accelerometer 212 for determining a frequency metric of the acceleration signal. The frequency metric may be compared to AT/AF detection criteria. When the frequency metric meets AT/AF detection criteria, control circuit 206 may compare the current count of AT/AF intervals and/or AT/AF events to a second threshold value for detecting AT/AF. When both the frequency metric and the AT/AF interval or event count meet detection criteria, processor 244 may detect an AT/AF episode. In other examples, the acceleration signal may be analyzed by processor 244 for determining when the acceleration signal meets AT/AF detection criteria without requiring the atrial electrical signal meeting AT/AF detection criteria or without requiring a count of AT/AF intervals or events to first reach a threshold count for triggering acceleration signal analysis for AT/AF detection.

Control circuit 206 may respond to an AT/AF detection by storing related data in memory 210. Additionally or alternatively, control circuit 206 may respond to the AT/AF detection by transmitting a signal via telemetry circuit 208 indicating that AT/AF is detected. Another medical device, e.g., ICD 112 of FIG. 3A, may respond to the transmitted signal by delivering a therapy to terminate the AT/AF or by withholding a VT/VF detection or a VT/VF therapy, as examples. In still other examples, control circuit 206 may respond to an AT/AF detection by controlling pulse generator 202 to deliver ATP therapy in some examples to overdrive pace the atria in an attempt to terminate the AT/AF. In still other examples, control circuit 206 may switch rate response control from being based on activity counts determined from the acceleration signal received from accelerometer 212 to being based on absolute or relative temperature changes determined from a temperature signal from temperature sensor 216.

Memory 210 may include computer-readable instructions that, when executed by control circuit 206, cause control circuit 206 to perform various functions attributed throughout this disclosure to pacemaker 14. The computer-readable instructions may be encoded within memory 210. Memory 210 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal.

Memory 210 may store AT/AF intervals determined from the atrial electrical signal and frequency metrics determined from the acceleration signal for use by processor 244 in detecting AT/AF. Memory 210 may store other data determined from sensed signals, e.g., patient physical activity metric and temperature data, by control circuit 206. Memory 210 may also store programmable control parameters and instructions executed by control circuit 206 for detecting AT/AF, controlling rate response pacing and other pacemaker functions.

Telemetry circuit 208 includes a transceiver 209 and antenna 211 for transmitting and receiving data, e.g., via a radio frequency (RF) communication link. Telemetry circuit 208 may be capable of bi-directional communication with external device 20 (FIG. 1) as described above. Acceleration signals, temperature signals, and cardiac electrical signals, and/or data derived therefrom, may be transmitted by telemetry circuit 208 to external device 20. Programmable control parameters and algorithms for sensing cardiac event signals, detecting AT/AF and controlling pacing therapies delivered by pulse generator 202 may be received by telemetry circuit 208 and stored in memory 210 for access by control circuit 206. AT/AF detection signals may be transmitted by telemetry circuit 208 for receipt by another medical device, e.g., ICD 112.

Power source 214 provides power to each of the other circuits and components of pacemaker 14 as required. Power source 214 may include one or more energy storage devices, such as one or more rechargeable or non-rechargeable batteries. Power source 214 provides power to activity sensing circuit 212 as required for operating accelerometer 212 and temperature sensor 216. For example, control circuit 206 may control when power is supplied to accelerometer 212 for use in determining a frequency metric for detecting AT/AF. When AT/AF is detected, control circuit 206 may control power supplied to temperature sensor 216 for producing a temperature signal and processing the temperature signal for controlling rate response pacing. When temperature is not needed, e.g., when AT/AF is not being detected, temperature sensor 216 may be powered down or powered for sampling the temperature signal at a relatively lower sampling rate to obtain a baseline, resting temperature signal. The connections between power source 214 and other pacemaker circuits and components are not explicitly shown in FIG. 4 for the sake of clarity but are to be understood from the general block diagram of FIG. 4. For example, power source 214 may provide power as needed to charging and switching circuitry included in pulse generator 202; amplifiers, ADC 226 and other components of sensing circuit 204; telemetry circuit 208 and memory 210.

The functions attributed to pacemaker 14 herein may be embodied as one or more processors, controllers, hardware, firmware, software, or any combination thereof. Depiction of different features as specific circuitry is intended to highlight different functional aspects and does not necessarily imply that such functions must be realized by separate hardware, firmware or software components or by any particular circuit architecture. Rather, functionality associated with one or more circuits described herein may be performed by separate hardware, firmware or software components, or integrated within common hardware, firmware or software components. For example, algorithms for determining AT/AF intervals from the atrial electrical signal, determining a frequency metric from the acceleration signal and detecting AT/AF may be implemented in control circuit 206 executing instructions stored in memory 210. Providing software, hardware, and/or firmware to accomplish the described functionality in the context of any modern medical device, given the disclosure herein, is within the abilities of one of skill in the art.

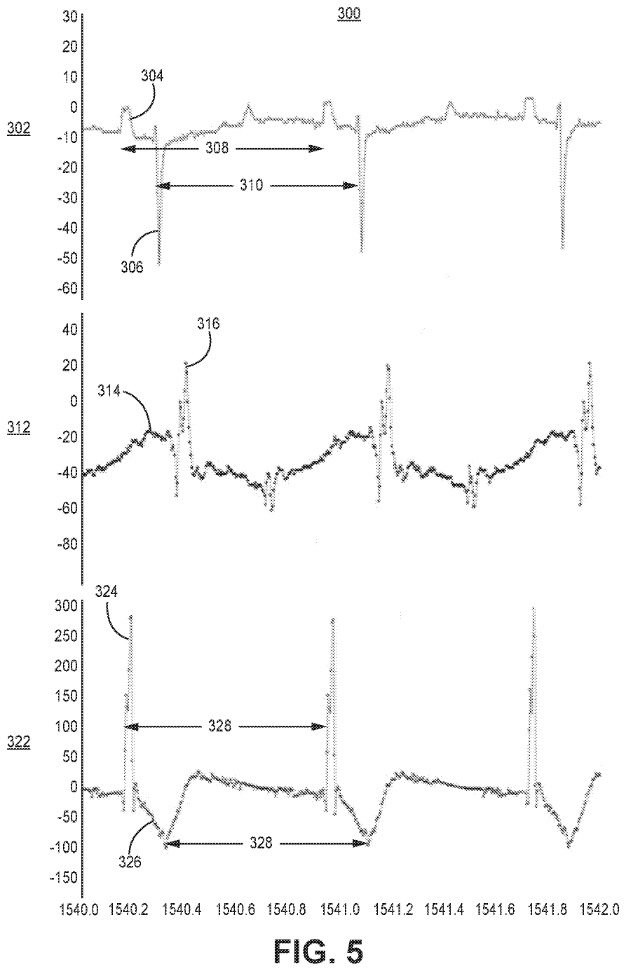

FIG. 5 is a diagram 300 of an electrocardiogram (ECG) signal 302 during normal sinus rhythm and a corresponding acceleration signal 312 and atrial electrogram (EGM) signal 322 that may be sensed by pacemaker 14. ECG signal 302 includes P-waves 304 followed by R-waves 306 occurring at regular PPIs 308 and RRIs 310, respectively. Atrial EGM signal 322 includes P-waves 324 and far-field R-waves (FFRWs) 326 occurring regularly at the respective PPIs 328 and RRIs 330. Acceleration signal 312 includes an acceleration signal 314 attendant to atrial contraction and corresponding in time to P-waves 324 and a relatively larger acceleration signal 316 attendant to ventricular contraction and corresponding in time to FFRWs 326.

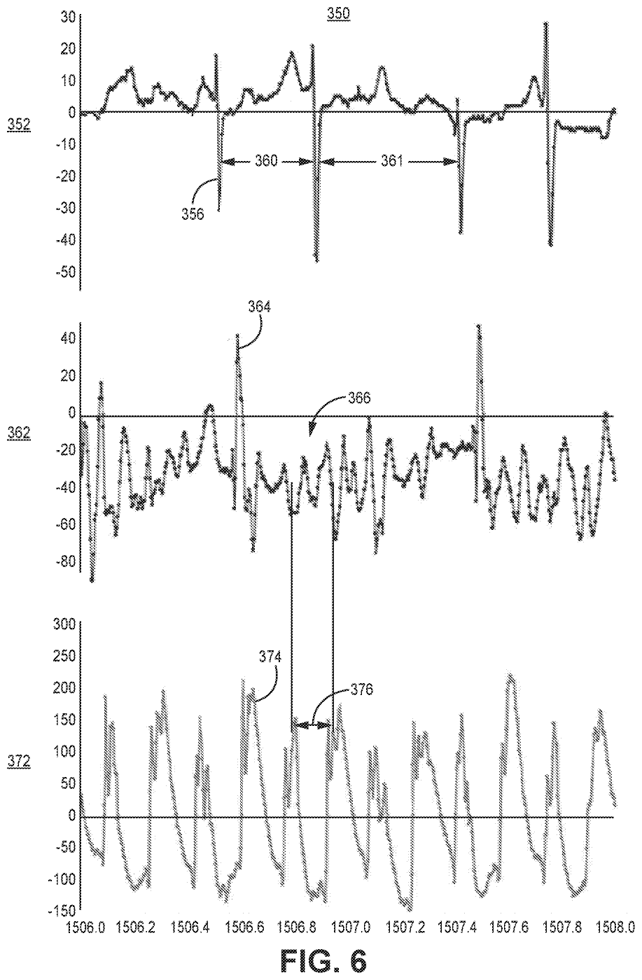

FIG. 6 is a diagram 350 of an ECG signal 352 during AF and a corresponding acceleration signal 362 and atrial EGM signal 372 that may be sensed by pacemaker 14. In the ECG signal 352, R-waves 356 and corresponding RRIs, e.g., 360 and 361, are irregular as some atrial fibrillation waves may conduct to the ventricles at irregular intervals. Generally, P-waves may be absent from the ECG signal during AF.

Atrial fibrillation waves 374 of the atrial EGM signal 372 are wide and occur at a fast rate during AF. FFRWs are not clearly observed in the atrial EGM signal 372 during AF. The atrial acceleration signal 362 includes some large amplitude acceleration signals 364 corresponding in time to some R-waves 356 in ECG 352. However, the acceleration signal 362 is characterized by high frequency oscillations 366, which occur at a higher frequency than the atrial fibrillation waves 374 of atrial EGM signal 372. The high frequency oscillations 366 of the acceleration signal 362 occur at approximately twice the frequency of atrial fibrillation waves 374, e.g., about two positive peaks in acceleration signal 362 occur for each PPI 376, determined as the time interval between two consecutively received atrial sensed event signals corresponding to two consecutive atrial fibrillation waves 374.

This increased frequency of oscillations of acceleration signal 362 during non-sinus AT/AF may be detected based on one or more frequency metrics determined by control circuit 206 from acceleration signal 362. Control circuit 206 may discriminate between sinus tachycardia and AT/AF based on the increased frequency of oscillations, approximately double the frequency of atrial fibrillation waves 374 (or atrial sensed event signals received from sensing circuit 204) since the frequency of oscillations of acceleration signals during sinus tachycardia may be expected to be similar to the rate of P-waves and atrial sensed event signals received from sensing circuit 204. As described below, control circuit 206 may be configured to determine a frequency metric from acceleration signal 362 that is correlated to the frequency of oscillations of the acceleration signal.

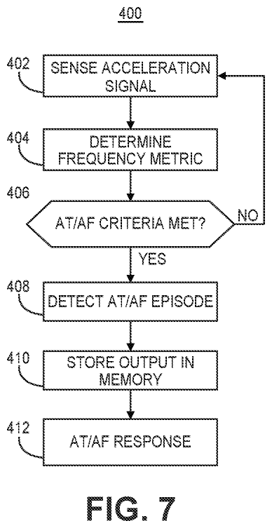

FIG. 7 is a flow chart 400 of a method for detecting AT/AF by pacemaker 14 according to some examples. At block 402, control circuit 206 enables accelerometer 212 to sense an acceleration signal. As described above, the acceleration signal may be single axis signal or the combination, e.g., a summation, of two or three axis signals. The acceleration signal may be filtered, amplified and digitized and in some examples rectified and passed to control circuit 206.

At block 404, processor 244 of control circuit 206 determines a frequency metric from the acceleration signal that is correlated to the frequency of oscillations of the acceleration signal or the number of oscillations, e.g., number of peaks, during a time interval. Various examples of methods that may be used by control circuit 206 for determining a frequency metric are described below. The frequency metric may be determined in the time domain or the frequency domain.

A time-frequency analysis may be performed to determine the frequency metric over multiple time intervals for detecting a high frequency of oscillations due to a relatively large increase in frequency during AT/AF compared to a sinus rhythm. The time frequency analysis may include performing a transform such as a spectrogram, Wavelet transform, Gabor transform, Wigner distribution, Gabor-Wigner transform or the like for determining the frequency content during a given time interval of the acceleration signal. The frequency metric may be determined as a characteristic frequency of the acceleration signal, e.g., a frequency having a maximum energy in the acceleration signal, a median or mean frequency component. The characteristic frequency determined from a time-frequency transform analysis is expected to be increased during AT/AF compared to a sinus rhythm, including sinus tachycardia.

In other examples, a frequency metric may be determined as a count of oscillations of the acceleration signal over a specified time interval or number of atrial cycles. The count of oscillations may be counted as the number of maximum peaks of the acceleration signal during a given time interval, which may be determined after rectifying the acceleration signal in some examples. The frequency metric determined as a count of oscillations may alternatively be determined as a count of the number of crossings of a specified threshold, which may include positive and/or negative threshold crossings. The threshold may be zero, for counting zero crossings, but may be set to another threshold having an absolute value greater than zero. The threshold crossings may be counted from the non-rectified or rectified acceleration signal.

In other examples, the frequency metric may be determined as a low slope content (LSC) by determining successive differences between sample points of the acceleration signal and comparing each successive difference to a low slope threshold. Each successive difference may be determined as the difference between two consecutive sample points of the acceleration signal or between two sample points separated by a specified number of one or more intervening sample points, e.g., the difference between the i^{th} sample point and the i-3 sample point as an example. When the difference between two successive sample points, is less than a low slope threshold, the slope of the acceleration signal is relatively low, indicating a high frequency oscillation is not likely occurring. The successive difference may be along a baseline portion of the acceleration signal. When the difference between two successive differences is greater than a low slope threshold, the relatively high slope is evidence of a possible high frequency oscillation. As such, the number of successive differences that are greater than the low slope threshold, which may be counted by control circuit 206 as a high slope content, may be correlated to the frequency of oscillations of the acceleration signal.

In one example, the LSC is determined as the ratio of the number of successive differences less than a low slope threshold to the total number of data points during an n-second time period. The low slope threshold may be defined as a percentage, for example 10%, 20%, 30%, 40% or other percentage of the largest absolute successive difference determined from the n-second signal segment. The LSC may then be determined by control circuit 206 as the number of successive differences having an absolute value less than the low slope threshold. A high value of the LSC indicates a high number of successive differences being less than the low slope threshold, which may be an indication of sinus rhythm. A low value of the LSC, indicating a high number of successive differences being greater than the low slope threshold, may indicate high frequency oscillations due to AT/AF.

In another example, the frequency metric may be determined by determining the rectified mean or median amplitude of the acceleration signal, the integration (summation) of the rectified acceleration signal, which may be normalized in some examples by a maximum or mean peak amplitude, a root mean square (determined by squaring each sample point amplitude, determining the mean and the square root of the mean) or other methods for determining the energy of the acceleration signal or a metric correlated to the energy of the acceleration signal. In each of these examples, the frequency metric may be inversely correlated to the amount of time the acceleration signal is at or near the baseline and is thus correlated to the frequency of oscillations. When AT/AF is occurring, the high frequency oscillations result in an acceleration signal that is not at or near the baseline amplitude except for zero crossings during each PPI. Accordingly, a high rectified mean or median amplitude, high integration value of the rectified signal, high root mean square or the like is an indication of a high frequency of oscillations during AT/AF since the amplitude of the acceleration signal is rarely at the baseline amplitude.

The frequency metric is not dependent on a maximum peak amplitude reached by the acceleration signal. Rather, the frequency metric is correlated (inversely or directly) to the amount of time the acceleration signal is not at or near the baseline amplitude and therefore correlated to the frequency of oscillations of the acceleration signal. In some examples, however, the sample point amplitudes used to determine the frequency metric or the final value of the frequency metric may be normalized by a maximum peak amplitude of the acceleration signal during the time interval over which the frequency metric is being determined. By normalizing by the maximum peak amplitude, occasional large amplitude waveforms, e.g., due to patient body motion, ventricular contraction or other large acceleration forces or noise, may not skew the resulting frequency metric. A threshold value may be defined and stored in memory 210 for discriminating between a relatively high rectified mean or median amplitude, integration value, or root mean square value that is likely associated with AT/AF from a relatively lower value of the respective frequency metric that is expected during a sinus rhythm, when the acceleration signal is more likely to be near a baseline value between cardiac mechanical event signals.

The frequency metric may be determined over a predetermined time interval, e.g., 0.25 seconds, 0.5 seconds, 1 second, 2 seconds, 3 seconds or other selected time interval. The time interval selected may depend, at least in part, on the frequency metric being determined. For example, the number of zero crossings, number of peaks, number of threshold crossings or time frequency transform may be performed over a time interval that is at least one to two seconds or more. The LSC, median amplitude, root mean square or other metrics may be determined over a relatively shorter time interval in some examples. The frequency metric may be determined over multiple consecutive time intervals for determining when AT/AF criteria are met.

Control circuit 206 may determine one or more of the frequency metrics described above for each time interval of multiple consecutive time intervals and classify each time interval as AT/AF or non-AT/AF based on a comparison of the frequency metric(s) to AT/AF criteria at block 406. When a threshold number of consecutive time intervals (or X of Y time intervals) are classified as AT/AF based on the frequency metrics, the AT/AF detection criteria are met at block 406. Control circuit 206 may detect AT/AF at block 408 based on the acceleration signal. In some examples, as described below, both the acceleration signal and the atrial electrical signal may be required to meet AT/AF criteria in order to detect AT/AF. For example, at least a threshold number of PPIs falling within an AT/AF interval zone may be required to detect AT/AF in addition to the acceleration signal AT/AF criteria being met.

At block 410, control circuit 206 may generate output that is stored in memory 210 relating to the AT/AF detection. The output may include the AT/AF detection with a corresponding date and time stamp, the duration of time of the AT/AF episode (e.g., based on the number of time intervals that met AT/AF criteria), or other data relating to the AT/AF detection. In some examples, a buffer in memory 210 may store the duration of each detected AT/AF episode and determine an AT/AF burden as the total accumulated time the patient is in AT/AF out of a 24 hour period or other time period (e.g., since time of implant of pacemaker 14).

At block 412, control circuit 206 may respond to the AT/AF detection by transmitting an AT/AF detection signal, storing AT/AF episode data for later transmission to external device 20, or adjusting a therapy delivered by pulse generator 202 as examples. Telemetry circuit 208 may transmit an AT/AF detection signal that may be received by another medical device, e.g., ICD 114. ICD 114 may respond to the transmitted AT/AF detection signal by withholding a VT or VF detection and therapy or delivering a cardioversion therapy to terminate the AT/AF. Control circuit 206 may respond to the AT/AF detection by controlling pulse generator 202 to deliver overdrive pacing or ATP therapy to terminate the AT/AF.

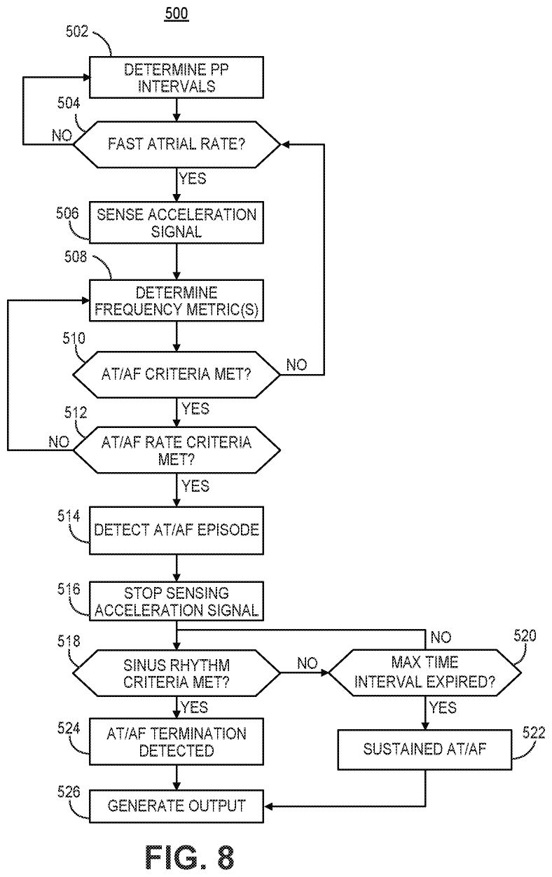

FIG. 8 is a flow chart 500 of a method for detecting AT/AF by atrial pacemaker 14 according to another example. At block 502, control circuit 206 receives atrial sensed event signals from sensing circuit 204. Control circuit 206 determines a PPI from one atrial sensed event signal to the next consecutive atrial sensed event signal. Control circuit 206 may compare each PPI to an AT/AF detection interval. The AT/AF detection interval may be programmable and tailored to a given patient and may be between 400 ms and 300 ms, as examples. For example, each PPI that is shorter than the AT/AF interval, e.g., shorter than 320 ms may be counted by control circuit 206 as an AT/AF interval.

At block 504, control circuit 206 may compare an AT/AF interval count to fast atrial rate criteria. The fast atrial rate criteria may require that the AT/AF interval count reach a threshold number of consecutive PPIs being AT/AF intervals, e.g., 3, 5, 8, 10, 15 or other threshold number of AT/AF intervals. In other examples, the fast atrial rate criteria may not require the threshold number of AT/AF intervals to be consecutive in order to detect a fast atrial rate at block 504. For instance, the fast atrial rate criteria may require 3 out 5, 5 out of 8, 8 out of 12, or other X AT/AF intervals out the most recent Y PPIs. In other examples a mean or median atrial rate over a predetermined number of most recent PPIs may be required to be greater than a threshold rate at block 504. The fast atrial rate criteria may be defined differently than rate-based AT/AF detection criteria applied to the sensed atrial electrical signal. As described below, the number of AT/AF intervals required to detect AT/AF after the fast atrial rate criteria are met may be higher than the fast atrial rate criteria requirements.

When a required percentage or number of PPIs are determined to be AT/AF intervals by control circuit 206, control circuit 206 may enable acceleration signal sensing and analysis at block 506. In some examples, a signal from the accelerometer may be sensed for determining a patient physical activity metric for providing rate response pacing. Accordingly, control circuit 206 may already be receiving an acceleration signal from accelerometer 212 when the fast atrial rate criteria are met at block 504. However, control circuit 206 may not be analyzing the acceleration signal for determining one or more frequency metrics correlated to the frequency of oscillations of the acceleration signal. In some examples, an acceleration signal received from accelerometer 212 for determining patient physical activity metrics for rate response pacing control may be received from a different accelerometer axis (or combination of axes) and/or undergo different filtering or other processing than the acceleration signal received from accelerometer 212 used for determining frequency metric(s) for AT/AF detection. As such, at block 506, control circuit 206 may enable sensing and analysis of the acceleration signal for use in AT/AF detection in response to determining that the fast atrial rate criteria are met at block 504.

At block 508, control circuit 206 determines one or more frequency metrics from the acceleration signal. As described above, control circuit 206 may perform a time-frequency transform, determine a count of maximum and/or minimum peaks or threshold crossings, determine a LSC, determine a median or mean amplitude, integration, root mean square and/or other metric or combination of metrics that is/are correlated to the frequency of acceleration signal oscillations. The frequency metric(s) may be determined over one or more specified time intervals or over one or more PPIs. Each frequency metric may be compared to AT/AF criteria at block 510.

The criteria applied at block 510 depends on the particular frequency metric(s) being determined. For example, when a time frequency transform or count of peaks or threshold crossings is determined, the resulting maximum, mean or median frequency or the resulting count may be compared to a threshold, which may be based on an analogous maximum, mean, median frequency or count value determined from the atrial electrical signal. A threshold indicating that the frequency of oscillations of the acceleration signal is at least 1.5 times the frequency of the sensed P-waves, for example, may be applied at block 510 for determining that AT/AF criteria are met. During sinus tachycardia, the frequency of oscillations of the acceleration signal are expected to match the rate of sensed atrial events. Therefore when the frequency metric corresponds to a frequency that is 1.5 times or higher, or two times or higher, than the frequency of sensed atrial event signals, non-sinus AT/AF is likely.

In some cases, FFRWs may be oversensed causing a fast atrial rate to be detected at block 504. However, when FFRWs are being oversensed during a sinus rhythm, the frequency of oscillations of the acceleration signal will not be higher than the atrial sensed event rate. As such, determination of the frequency metric enables control circuit 206 to determine when a fast rate may be due to oversensing of FFRWs based on a frequency metric determined from the acceleration signal having a relatively lower value, corresponding to the true atrial rate.

In other examples, an LSC threshold, a median amplitude threshold, mean amplitude threshold, root mean square threshold or integration threshold may be defined that discriminates the frequency of oscillations of atrial acceleration signals due to atrial contraction during sinus rhythm from the relatively high frequency oscillations of the acceleration signal during AT/AF.

When the AT/AF criteria are not met by the acceleration signal (“no” branch of block 510), control circuit 206 may verify that a fast atrial rate is still being detected at block 504 and, if so, continue analysis of the acceleration signal for determining when AT/AF criteria are met. If a fast atrial rate is no longer being detected, control circuit 206 may return to block 502 to continue monitoring PPIs for AT/AF intervals.

When AT/AF criteria are met by the acceleration signal at block 510, control circuit 80 may verify that rate or interval based AT/AF criteria are satisfied at block 512 as determined from the atrial electrical signal. For example, AT/AF rate detection criteria may specify a required number of AT/AF intervals to detect (sometimes referred to as number of intervals to detect or “NID”). An NID of 18, 24, 28, 32 or other specified number of AT/AF intervals may be required. The required number of AT/AF intervals may or may not be required to be consecutive. For example, 18 out of 22, 20 out of 24, 24 out of 32 or other N of M criteria may be specified or programmed as AT/AF rate criteria applied to the sensed electrical events at block 512.

If the AT/AF rate criteria are not yet met at block 512, e.g., when an AT/AF NID has not yet been reached, control circuit 206 may return to block 508 to continue analyzing the acceleration signal. If the acceleration signal no longer meets the AT/AF criteria at block 510, control circuit 206 may return to block 504 to check if a fast atrial rate is still being detected. When the acceleration signal satisfies the AT/AF criteria at block 510 and the atrial electrical signal satisfies the AT/AF rate criteria at block 512, e.g., an NID is reached or mean or median atrial rate meets AT/AF rate criteria, control circuit 206 detects AT/AF at block 514.

At block 516, control circuit 206 may optionally disable the accelerometer or at least disable some or all processing and analysis of the accelerometer signal in some examples. Once AT/AF is detected, the atrial electrical signal may be analyzed at block 518 to detect when the atrial rate, based on PPIs, is less than an AT/AF rate. Control circuit 206 may apply sinus rhythm criteria to the PPIs determined between consecutively received atrial sensed event signals. For example, when a threshold number of PPIs (consecutive or X of Y PPIs) are longer than an AT/AF termination interval, termination of the AT/AF episode may be detected by control circuit 206 at block 524. The AT/AF termination interval applied to PPIs to detect termination may be equal to or greater than the AT/AF detection interval applied at block 512 to detect AT/AF to allow for hysteresis (e.g., for avoiding frequent redetections of the same AT/AF episode). The number of PPIs required to be longer than the AT/AF termination interval to detect termination may be higher, lower or equal to the number of AT/AF intervals required to detect AT/AF.

In other examples, AT/AF termination may be detected at block 524 based on a running mean or median PPI or a minimum PPI determined from a predetermined number of PPIs buffered in memory 210. For example, control circuit 206 may determine ten, sixteen, twenty or other specified number of consecutive PPIs and determine the mean, median or minimum PPI from the buffered PPIs. The mean, median or minimum PPI may be compared to the AT/AF termination interval at block 518. When the mean, median or minimum PPI is greater than the AT/AF termination interval, sinus rate criteria are met and AT/AF termination is detected at block 524.

While not explicitly shown in FIG. 8 but as described above in conjunction with FIG. 7, it is to be understood that in some examples the pacemaker pulse generator 202 may deliver a pacing therapy to terminate the AT/AF episode upon AT/AF detection at block 514. In other examples, telemetry circuit 208 may transmit an AT/AF detection signal at block 514 and another device, e.g., ICD 114 shown in FIG. 3A, may deliver a therapy to terminate the AT/AF episode. In other instances, the AT/AF episode may spontaneously terminate.

In some cases, the AT/AF episode may be sustained. When sinus rhythm criteria are not met at block 518 based on an analysis of the PPIs, control circuit 206 may determine if a maximum time interval has expired at block 520 and, if so, detect a sustained AT/AF episode at block 522. For example, if AT/AF termination is not detected after one minute, five minutes, ten minutes or other specified time interval, a sustained AT/AF episode may be detected at block 522. Control circuit 206 may generate an output at block 526 to store the sustained AT/AF detection in memory 210, record atrial electrical signal and/or acceleration signal episode in memory 210, and/or transmit a clinician and/or patient notification indicating that a sustained AT/AF episode is detected and medical attention may be needed.

During AT/AF, the acceleration signal may be unreliable for use in determining a patient physical activity metric and controlling rate response pacing. As such, control circuit 206 may disable acceleration signal sensing as indicated at block 516 of FIG. 8. As described below in conjunction with FIG. 11, control circuit 206 may enable rate response pacing control based on the temperature signal received from temperature sensor 216. As such, to conserve power source 214, acceleration signal sensing or at least acceleration signal analysis may be disabled since the atrial electrical signal is expected to be reliable when a slower, sinus rate returns. Using the atrial electrical signal for detecting AT/AF termination instead of the acceleration signal may improve the overall longevity of the pacemaker 14. However, it is contemplated that the acceleration signal sensing and analysis may remain enabled (omitting block 516) or may be intermittently enabled after detecting AT/AF (temporarily disabled at block 514) for use in detecting AT/AF termination.

Frequency metric(s) determined from the acceleration signal may be compared to sinus rhythm criteria at block 518. When the frequency metric meets a threshold requirement, for example, that is indicative of a frequency of oscillations that approximately matches the rate of sensed atrial event signals (or is less than twice or less than 1.5 times the frequency of atrial sensed event signals), sinus rhythm criteria may be met at block 518. The sinus rhythm criteria applied at block 518 may include criteria applied to one or more frequency metrics determined from the acceleration signal and/or criteria applied to PPIs determined from the atrial electrical signal. Furthermore, it is contemplated that morphology analysis of the atrial electrical signal may be performed to detect a transition from AT/AF waveforms to sinus P-waves in addition to or instead of the acceleration signal and/or atrial rate (PPI) analysis described above.

At block 526, control circuit 206 may generate an output, which may be stored in memory 210, in response to detecting AT/AF episode. The output may be an AT/AF detection flag with associated data relating to the AT/AF episode, such as a date and time stamp, episode duration and cumulative AT/AF burden, and/or a recording of the atrial electrical signal with atrial sensed event markers and the atrial acceleration signal. The AT/AF episode data may be transmitted to external device 20 for review by a clinician. While control circuit 206 is shown to generate output at block 526 after detecting AT/AF termination in FIG. 8, it is to be understood that control circuit 206 may generate an output such as delivering a pacing therapy or transmitting an AT/AF detection notification signal, as described above, in response to AT/AF detection, prior to detecting termination.

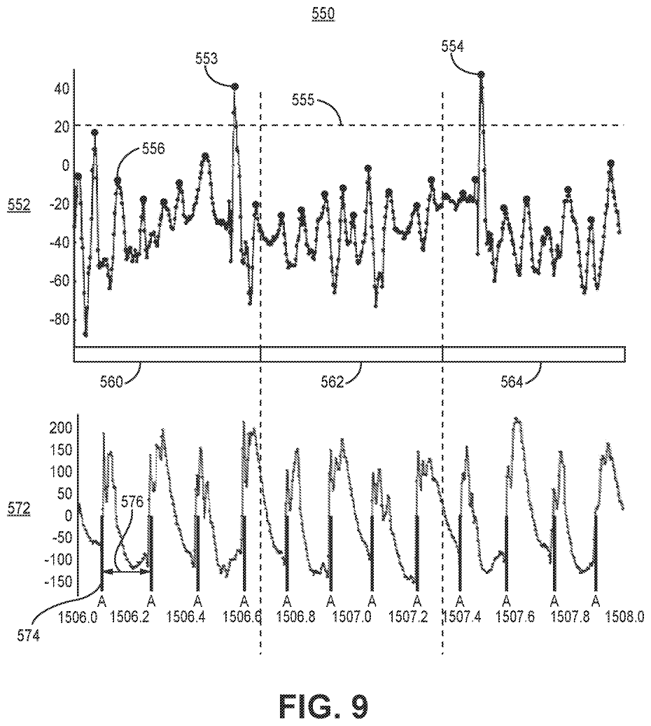

FIG. 9 is a diagram 550 of an acceleration signal 552 and atrial EGM signal 572 illustrating one method that may be executed by control circuit 206 for determining a frequency metric from acceleration signal 552. Atrial sensed event signals 574 may be generated by sensing circuit 204 in response to the rectified atrial electrical signal crossing a P-wave sensing threshold. Control circuit 206 may determine PPIs 576 between each consecutive pair of atrial sensed event signals 574. As described above in conjunction with FIG. 8, control circuit 206 may compare each PPI 576 to an AT/AF threshold interval to count AT/AF intervals and determine when fast atrial rate criteria are met at block 504. For instance, when a threshold number of PPIs 576 are shorter than an AT/AF threshold interval, control circuit 206 may begin processing and analysis of acceleration signal 552 for determining a frequency metric for detecting AT/AF.

Control circuit 206 may set time intervals 560, 562 and 564. At least one frequency metric may be determined from the acceleration signal 552 that is sensed during each respective time interval 560, 562 and 564. In the example shown, control circuit 206 determines a count of acceleration signal oscillations by counting the positive maximum peaks 556 from the acceleration signal 552 during each time interval 560, 562 and 564. The highest maximum peaks 553 and 554 may correspond to ventricular contractions as described above in conjunction with FIG. 6. In some examples, control circuit 206 may determine each maximum peak amplitude and compare the amplitude to a threshold amplitude 555. Any maximum peaks 553 and 554 that are greater than the threshold amplitude 555 may be rejected as non-atrial events and not counted by control circuit 206.

Control circuit 206 may include a peak track and hold circuit for detecting the maximum peaks 556. Control circuit 206 may increment a peak counter each time a maximum peak is detected without a positive-going crossing of threshold amplitude 555 by acceleration signal 552 since the most recent preceding counted maximum peak. When a positive going crossing of threshold amplitude 555 is detected, control circuit 206 may wait for the acceleration signal to be less than the threshold amplitude 555 before counting the next detected maximum peak.

The count of maximum peaks 556 reached during each time interval 560, 562 and 564 may be buffered in memory 210 for a predetermined number of time intervals. Each count may be compared to a threshold count to classify each time interval 560, 562 and 564 as an AT/AF interval or a sinus interval (non-AT/AF interval). The threshold count may be predetermined and based on the minimum number of AT/AF intervals that could occur within the respective time interval. The threshold count may be set to 1.5, 1.6, 1.8 or other multiple of the minimum number of AT/AF intervals that could occur within each time interval, for example. For instance, if the AT/AF threshold interval is 320 ms and each interval 560, 562 and 564 is 1 second long, a minimum of three AT/AF intervals is expected during each time interval. More than four maximum peaks are expected to be counted during each time interval in order to detect 1.5 times or higher frequency of oscillations of the acceleration signal. In other examples, the threshold count may be set based on the actual number of atrial sensed event signals 574 counted by control circuit 206 during the respective time interval 560, 562 or 564.

In the example shown, in time interval 560, control circuit 206 receives four atrial sensed event signals 574. Control circuit 206 may determine the peak count threshold to be 1.5 times this number of atrial sensed event signals or 6. Control circuit 206 determines a count of eight maximum positive peaks in time interval 560, excluding the maximum peak 553 exceeding threshold amplitude 555. Since the count of maximum positive peaks is greater than 1.5 times the number of atrial sensed event signals, control circuit 206 may classify time interval 560 as AT/AF. In this example, the frequency of oscillations of the acceleration signal is approximately twice the frequency of atrial sensed event signals, which is evidence of AT/AF.

Similarly, at the end of the next time interval 562, control circuit 206 reaches a count of nine maximum positive peaks of acceleration signal 552 and a count of four atrial sensed event signals. At the end of time interval 564, the acceleration signal maximum peak count is nine (excluding maximum peak 554), and the atrial sensed event count is four. Control circuit 206 may classify time intervals 562 and 564 as AT/AF intervals based on the frequency metric of the acceleration signal indicating a frequency of oscillations of more than 1.5 times (approximately twice) the frequency of atrial sensed event signals 574.

In other examples, control circuit 206 may set the threshold amplitude 555 lower, or set a second lower threshold amplitude, and count the number of the lower threshold crossings during each time interval 560, instead of counting the number of maximum peaks, to determine the frequency metric correlated to the frequency of oscillations of acceleration signal 552. In still other examples, control circuit 206 may determine a mean or median acceleration signal amplitude from all of the sample points of acceleration signal 552 spanning each time interval 560, 562 and 564 and compare the mean or median amplitude to a threshold amplitude that discriminates between higher frequency of oscillations during AT/AF and the lower frequency of oscillations during sinus rhythm.

Other examples of frequency metrics that may be determined over each time interval 560, 562 and 564 are described above, including time-frequency transform for determining a highest energy frequency, the LSC, integration of the rectified acceleration signal, root mean square, etc. Each of these frequency metrics correlated to the number or frequency of oscillations of acceleration signal 552 during a given time interval may be compared to a threshold or criteria that discriminates from the relatively lower frequency of oscillations occurring during sinus tachycardia, normal sinus rhythm, bradycardia, a paced atrial rhythm or other non-AT/AF rhythm.

Control circuit 206 may determine that the AT/AF criteria are met at block 510 of FIG. 5 when a threshold number of consecutive or non-consecutive (X of Y) time intervals are classified as AT/AF intervals. For instance, all three time interval 560, 562 and 564 may be required to be classified as AT/AF intervals or two out three may be required to be classified as AT/AF intervals for AT/AF criteria to be met at block 510 of FIG. 8.

The time intervals 560, 562 and 564 may be fixed intervals ranging from 0.25 seconds to 10 seconds, or from 1 to 3 seconds, as examples. In the example shown, each time interval is approximately 0.6 to 0.8 seconds. The selected time interval may be set to a multiple of the programmed AT/AF detection interval in some examples. In other examples, the time intervals may be variable and be started upon receiving an atrial sensed event signal 574 and terminated on the nth atrial sensed event signal such that each time interval is defined by a fixed number of PPIs. Control circuit 206 may determine one or more frequency metrics, including any of the examples described above, for at least one time interval to classify the at least one time interval as AT/AF or as non-AT/AF. The time intervals 560, 562 and 564 may be continuously consecutive with no intervening delay as shown in FIG. 9. In other examples, the time intervals 560, 562 and 564 may be spaced apart time intervals, e.g., with a fraction of a second or one or more seconds between each time interval, such that frequency metrics are determined at spaced apart sampling time intervals. In still other examples, frequency metrics may be determined for predetermined time intervals that may be overlapping time intervals rather than consecutive time intervals as shown in FIG. 9.

In some examples, control circuit 206 may determine one or more frequency metrics during first time intervals having a first duration and determine or more frequency metrics during second time intervals having a second duration different than the first duration. For example, control circuit 206 may determine the maximum positive peak count during time intervals 560, 562 and 564 as described above, where each time interval is approximately 700 ms to 1.5 seconds, as examples. Control circuit 206 may additionally determine an integration of the rectified acceleration signal over a longer time interval, e.g., a two to three second time interval. Control circuit 206 may classify each of the shorter, first time intervals by comparing the maximum peak count to a threshold count and classify each of the longer, second time intervals by comparing the integration value to a threshold integration value. The number of shorter time intervals and maximum peak amplitude counts determined by control circuit 206 may be different than the number of longer time intervals and integration values determined by control circuit 206. Control circuit 206 may determine that the AT/AF criteria are met by acceleration signal 552 when the number of first time intervals classified as AT/AF reaches a first threshold number of AT/AF intervals and/or the number of second time interval classified as AT/AF reaches a second threshold number of AT/AF intervals. For example, control circuit 206 may determine that AT/AF criteria are met at block 510 when the maximum peak amplitude count for three, one-second intervals is at least 1.5 times the number of atrial sensed event signals and when the integration value of the rectified acceleration signal over one, three-second interval is greater than a threshold integration value. It is recognized that numerous combinations of different frequency metrics described herein, which may be determined over different time intervals, and corresponding AT/AF detection criteria may be defined to detect when the acceleration signal includes oscillations occurring at a frequency that is greater than the frequency of atrial sensed event signals, e.g., 1.5 to 2 times or more than the frequency of atrial sensed event signals.

FIG. 10 is a flow chart 600 of a method for detecting and responding to an AT/AF detection by pacemaker 14 according to another example. In some examples, pacemaker 14 is configured to provide rate response pacing. At block 602, control circuit 206 determines a patient physical activity metric from the acceleration signal received from accelerometer 212. As described above, the activity metric may be determined by summing acceleration signal sample point amplitudes over every two second time interval to obtain an “activity count.” The activity metric may be converted to a sensor indicated pacing rate (SIR) by control circuit 206 at block 603 according to a transfer function that relates the activity count to a target pacing rate.

At block 604, control circuit 206 may determine at least one frequency metric from the acceleration signal according to any of the example techniques described above. The acceleration signal frequency metric may be determined at block 604 only when the atrial rate is determined to be a fast rate in some examples. For example, when the PPIs satisfy fast rate criteria as described above. In other examples, the frequency metric may be determined at block 604 when the activity metric or corresponding SIR is greater than a predetermined threshold, e.g., corresponding to a relatively high level of patient physical activity such as greater than activities of daily living. In this way, the AT/AF criteria may be applied to the acceleration signal when the patient physical activity metric is relatively high to avoid adjusting the pacing rate when the high patient physical activity metric is caused by increased acceleration signal oscillations during AT/AF.

The frequency metric(s) are compared to AT/AF criteria at block 606. When AT/AF criteria are not met, control circuit 206 may adjust the atrial pacing rate toward the SIR at block 610. As long as the frequency of oscillations of the acceleration signal do not meet AT/AF criteria, the acceleration signal is deemed reliable for controlling rate response pacing. Control circuit 206 may adjust the pacing rate toward the SIR at block 610, which may be an increase, decrease, or no change in the pacing rate. The actual pacing rate may be adjusted according to a maximum pacing rate acceleration/deceleration limit toward the SIR.

When the frequency metric meets AT/AF criteria at block 606, the accelerometer 212, or at least acceleration signal processing and analysis, may be disabled at block 612. During AT/AF, the increased oscillations of the acceleration signal may contribute to the patient physical activity metric, artificially causing the SIR to be increased greater than the actual metabolic demand of the patient. As such, when the acceleration signal meets the AT/AF criteria, control circuit 206 may conserve power source 214 by disabling the accelerometer 212. Once the AT/AF criteria are met, AT/AF detection may be made as described above, based solely on the acceleration signal or upon rate-based AT/AF detection criteria being met in addition to the acceleration signal meeting the AT/AF criteria. Control circuit 206 may enable analysis of the temperature signal from temperature sensor 216 at block 614. The absolute temperature or a relative temperature change may be determined by control circuit 206 at block 614 for controlling rate response pacing. For example, temperature values may be buffered in memory 210 at predetermined time intervals, e.g., every 10 seconds, every 30 seconds, every minute, or every five minutes as examples. The temperature change may be determined as the difference in in two consecutively buffered temperature values.

Based on the temperature change, control circuit 206 adjusts the rate response pacing rate at block 616. When the temperature change is increasing, control circuit 206 may increase the pacing rate according to a maximum pacing rate acceleration limit. When the temperature is decreasing, control circuit 206 may decrease the pacing rate according to a pacing rate deceleration limit. When temperature is not changing, control circuit 206 may hold the pacing rate the same. Other example techniques that may be used by control circuit 206 for controlling rate response pacing based at least in part on the temperature sensor signal when the acceleration signal may be unreliable are generally disclosed in provisional U.S. patent application Ser. No. 63/076,420, filed Sep. 10, 2020 (Yoon, et al.), and subsequently filed U.S. patent application Ser. No. 17/404,517 filed Aug. 17, 2021 (Yoon, et al.), incorporated herein by reference in their entirety.

The pulse generator 202 may or may not be delivering pacing pulses at the rate response pacing rate set by control circuit 206 based on temperature at block 616 or based on the acceleration signal at block 610. Pace timing circuit 242 may set the atrial pacing escape interval timer according to the rate response pacing rate set by control circuit 206. Pulse generator 202 generates an atrial pacing pulse at the rate response pacing rate when the escape interval timer expires without receiving an atrial sensed event signal. If AT/AF is detected, the pacing escape interval is unlikely to expire without sensing an atrial event and restarting the pacing escape interval. In some instances, however, atrial fibrillation waves or low amplitude P-waves may be undersensed by sensing circuit 204 during AT/AF such that an occasional pacing pulse may be generated and delivered by pulse generator 202. Furthermore, when AT/AF terminates, the rate response pacing interval set according to the temperature signal permits rate response pacing as needed at an appropriate rate and allows for a smooth transition from the temperature-based rate response pacing rate toward the SIR based on the activity metric determined from the acceleration signal when acceleration signal sensing and analysis is re-enabled.

At block 618, control circuit 206 may determine PPIs based on the atrial sensed event signals received from sensing circuit 204 and/or delivered pacing pulses. The PPIs may be used by control circuit 206 to detect termination of the AT/AF episode as described above in conjunction with FIG. 8. Control circuit 206 may detect termination based on a threshold number of PPIs greater than the AT/AF detection interval, a mean or median PPI greater than the AT/AF detection interval, or pacing delivered at the rate response pacing rate. If termination is not detected, control circuit 206 continues to analyze the temperature signal at block 614 for controlling the rate response pacing rate and monitoring the atrial electrical signal for detecting AT/AF termination.

When termination is detected at block 620, control circuit 206 may re-enable acceleration signal analysis by powering on the accelerometer at block 622 and begin processing and analyzing the acceleration signal for use in controlling the rate response pacing rate by returning to block 602. The acceleration signal may also be processed and analyzed for detecting AT/AF as needed, e.g., when a fast atrial rate is detected and/or when a relatively high patient physical activity metric is determined, which may be artificially high due to atrial wall oscillations contributing to the patient physical activity metric during AT/AF. Upon re-enabling acceleration signal sensing and analysis, the rate response pacing rate may transition from being adjusted according to temperature change to being adjusted toward the SIR determined from the activity metric at block 602.

While the accelerometer signal sensing or analysis and processing of the accelerometer signal are shown to be disabled (block 612) and enabled (block 622), e.g., to conserve power source 214, it is to be understood that disabling acceleration signal sensing or analysis is optional. The acceleration signal may continue to be sensed after AT/AF criteria are met and analysis may be performed, e.g., for monitoring for AT/AF termination and/or for updating a SIR, but the activity metrics and SIR rate (if determined) may be ignored by control circuit 80 for the purposes of controlling rate response pacing. The first activity metric determined after AT/AF termination is detected may be used to update the SIR and transition from the temperature-based rate response rate toward the updated target SIR rate.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

What is claimed is:

1. A medical device comprising:
an accelerometer configured to sense an acceleration signal;
a control circuit configured to:
receive the acceleration signal;
determine from the acceleration signal at least one frequency metric that is correlated to a frequency of oscillations of the acceleration signal;
determine that the at least one frequency metric meets atrial tachyarrhythmia criteria; and
detect an atrial tachyarrhythmia in response to at least the frequency metric meeting the atrial tachyarrhythmia criteria.

2. The medical device of claim 1, wherein the control circuit is configured to:
determine the at least one frequency metric by:
performing a time-frequency transform of the acceleration signal;
determining a characteristic frequency of the acceleration signal based on the time-frequency transform; and
determine that the frequency metric meets atrial tachyarrhythmia criteria by determining that the characteristic frequency is greater than a frequency threshold.

3. The medical device of claim 1, wherein the control circuit is configured to:
determine the at least one frequency metric by:
setting a time interval; and
determining a count of acceleration signal oscillations during the time interval; and
determine that the frequency metric meets atrial tachyarrhythmia criteria by determining that the count of acceleration signal oscillations is greater than a threshold value.

4. The medical device of claim 1, wherein the control circuit is configured to:
determine the at least one frequency metric by:
setting a time interval; and
determining at least one of a low slope content, an integrated value, a median amplitude, a mean amplitude or a root mean square of the acceleration signal sensed over the time interval.

5. The medical device of claim 1, further comprising:
a cardiac electrical signal sensing circuit configured to:
sense a cardiac electrical signal; and
generate atrial sensed event signals in response to P-wave sensing threshold crossings by the cardiac electrical signal;
wherein the control circuit is further configured to:
receive the atrial sensed event signals;
determine that fast atrial rate criteria are met based on the atrial sensed event signals; and
determine the at least on frequency metric from the acceleration signal in response to the fast atrial rate criteria being met.

6. The medical device of claim 1, further comprising:
a cardiac electrical signal sensing circuit configured to:
sense a cardiac electrical signal; and
generate atrial sensed event signals in response to P-wave sensing threshold crossings by the cardiac electrical signal;
wherein the control circuit is further configured to:
receive the atrial sensed event signals generated by the cardiac electrical signal sensing circuit;
determine a frequency metric threshold based on a frequency of the atrial sensed event signals; and
determine that the atrial tachyarrhythmia criteria are met in response to the frequency metric being greater than the frequency metric threshold.

7. The medical device of claim 1, further comprising:
a cardiac electrical signal sensing circuit configured to:
sense a cardiac electrical signal; and
generate atrial sensed event signals in response to P-wave sensing threshold crossings by the cardiac electrical signal;
wherein the control circuit is further configured to:
receive the atrial sensed event signals;
disable the accelerometer in response to detecting the atrial tachyarrhythmia;
determine that termination criteria are met based on the atrial sensed event signals; and
detect termination of the atrial tachyarrhythmia episode in response to the termination criteria being met.

8. The medical device of claim 1, further comprising a temperature sensor configured to sense a temperature signal;
wherein the control circuit is further configured to:
determine a patient physical activity metric based on the acceleration signal;
determine a rate response pacing rate based on the patient physical activity metric; and
responsive to determining that the atrial tachyarrhythmia criteria are met, adjust the rate response pacing rate based on the temperature signal.

9. The medical device of claim 1, wherein the control circuit is further configured to:
determine the at least one frequency metric from the acceleration signal for each one of a plurality of time intervals;
classify each one of the plurality of time intervals as one of an atrial tachyarrhythmia time interval or a non-atrial tachyarrhythmia time interval based on the frequency metrics; and
determine that the at least one frequency metric meets the atrial tachyarrhythmia criteria in response to determining that a threshold number of the plurality of time intervals are classified as atrial tachyarrhythmia time intervals.

10. The medical device of claim 1, wherein the control circuit is further configured to:
determine a first frequency metric from the acceleration signal sensed during a first time interval having a first duration, the first frequency metric correlated to a frequency of oscillations of the acceleration signal;
determine a second frequency metric from the acceleration signal sensed during a second time interval having a second duration different than the duration of the first time interval, the second frequency metric different than the first frequency metric; and
determine that the first frequency metric and the second frequency metric meet the atrial tachyarrhythmia criteria.

11. The medical device of claim 1, further comprising a pulse generator configured to generate pacing pulses according to a pacing therapy in response to the control circuit detecting that atrial tachyarrhythmia.

12. The medical device of claim 1, further comprising a telemetry circuity configured to transmit an atrial tachyarrhythmia detection notification in response to the control circuit detecting the atrial tachyarrhythmia.

13. The medical device of claim 1, further comprising:
a pulse generator;
a housing enclosing the accelerometer, the control circuit, and the pulse generator, the housing comprising a pair of housing-based electrodes coupled to the pulse generator.

14. A method comprising:
sensing an acceleration signal;
determining at least one frequency metric from the acceleration signal that is correlated to a frequency of oscillations of the acceleration signal;
determining that the at least one frequency metric meets atrial tachyarrhythmia criteria; and
detecting an atrial tachyarrhythmia in response to at least the frequency metric meeting the atrial tachyarrhythmia criteria.

15. The method of claim 14, wherein:
determining the at least one frequency metric comprises:
performing a time-frequency transform of the acceleration signal;
determining a characteristic frequency of the acceleration signal based on the time-frequency transform; and
determining that the frequency metric meets atrial tachyarrhythmia criteria comprises determining that the characteristic frequency is greater than a frequency threshold.

16. The method of claim 14, wherein:
determining the at least one frequency metric comprises:
setting a time interval; and
determining a count of acceleration signal oscillations during the time interval; and
determining that the frequency metric meets atrial tachyarrhythmia criteria comprises determining that the count of acceleration signal oscillations is greater than a threshold value.

17. The method of claim 14, wherein:
determining the at least one frequency metric comprises:
setting a time interval; and
determining at least one of a low slope content, an integrated value, a median amplitude, a mean amplitude or a root mean square of the acceleration signal sensed over the time interval.

18. The method of claim 14, further comprising:
sensing a cardiac electrical signal;
generating atrial sensed event signals in response to P-wave sensing threshold crossings by the cardiac electrical signal;
determining that fast atrial rate criteria are met based on the atrial sensed event signals; and
determining the at least on frequency metric from the acceleration signal in response to the fast atrial rate criteria being met.

19. The method of claim 14, further comprising:
sensing a cardiac electrical signal;
generating atrial sensed event signals in response to P-wave sensing threshold crossings by the cardiac electrical signal;
determining a frequency metric threshold based on a frequency of the atrial sensed event signals; and
determining that the atrial tachyarrhythmia criteria are met in response to the frequency metric being greater than the frequency metric threshold.

20. The method of claim 14, further comprising:
sensing a cardiac electrical signal;
generating atrial sensed event signals in response to P-wave sensing threshold crossings by the cardiac electrical signal;
disabling the accelerometer in response to detecting the atrial tachyarrhythmia;
determining that termination criteria are met based on the atrial sensed event signals; and
detecting termination of the atrial tachyarrhythmia episode in response to the termination criteria being met.

21. The method of claim 14, further comprising:
determining a patient physical activity metric based on the acceleration signal;
determining a rate response pacing rate based on the patient physical activity metric;
sensing a temperature signal; and
responsive to determining that the atrial tachyarrhythmia criteria are met, adjusting the rate response pacing rate based on the temperature signal.

22. The method of claim 14, further comprising:
determining the at least one frequency metric from the acceleration signal for each one of a plurality of time intervals;
classifying each one of the plurality of time intervals as one of an atrial tachyarrhythmia time interval or a non-atrial tachyarrhythmia time interval based on the frequency metrics; and
determining that the at least one frequency metric meets the atrial tachyarrhythmia criteria in response to determining that a threshold number of the plurality of time intervals are classified as atrial tachyarrhythmia time intervals.

23. The method of claim 14, further comprising:
determining a first frequency metric from the acceleration signal sensed during a first time interval having a first duration, the first frequency metric correlated to a frequency of oscillations of the acceleration signal;
determining a second frequency metric from the acceleration signal sensed during a second time interval having a second duration different than the first duration of the first time interval, the second frequency metric different than the first frequency metric; and
determining that the first frequency metric and the second frequency metric meet the atrial tachyarrhythmia criteria.

24. The method of claim 14, further comprising generating pacing pulses according to a pacing therapy in response to detecting that atrial tachyarrhythmia.

25. The method of claim 14, further comprising transmitting an atrial tachyarrhythmia detection notification in response to detecting the atrial tachyarrhythmia.

26. A non-transitory, computer-readable storage medium storing a set of instructions which, when executed by a control circuit of a medical device, cause the medical device to:
sense an acceleration signal;
determine at least one frequency metric from the acceleration signal that is correlated to a frequency of oscillations of the acceleration signal;
determine that the at least one frequency metric meets atrial tachyarrhythmia criteria; and
detect an atrial tachyarrhythmia in response to at least the frequency metric meeting the atrial tachyarrhythmia criteria.
